(19) United States
(12) Patent Application Publication
(10) Pub. No.: US 2026/0345191 A1
(43) Pub. Date: Nov. 5, 2026

(54) Nerve Monitoring Integration With An Ultrasonic Surgical System

(71) Applicant: Stryker European Operations Limited, Carrigtwohill, Co Cork (IE)

(72) Inventors: Stephen Faul, Cork (IE); Sean McMahon, Dublin (IE)

(73) Assignee: Stryker European Operations Limited, Carrigtwohill, Co Cork (IE)

(21) Appl. No.: 19/118,899

(22) PCT Filed: Oct. 9, 2023
(86) PCT No.: PCT/IB2023/060135

§ 371 Date: Apr. 7, 2025

Related U.S. Application Data
(60) Provisional application No. 63/378,752, filed on Oct. 7, 2022.

(51) Int. Cl. A61B 17/32; A61B 17/00

(52) U.S. Cl. CPC A61B 17/320068; A61B 2017/00199; A61B 2017/320084

(57) ABSTRACT

An ultrasonic handpiece assembly is provided. The ultrasonic handpiece assembly includes a housing, an electromechanical transducer at least partially disposed within the housing, a tip operatively coupled to the electromechanical transducer, an electrical cord having a console connector, and a line connector. The electrical cord includes a first conductor, the first conductor being in electrical communication with the electromechanical transducer. The line connector includes a first tubing coupler configured to receive an irrigation line, a second tubing coupler configured to receive an aspiration line, and a receptacle including a first terminal, the receptacle being configured to receive a line extending from a nerve monitor, wherein the first terminal is in electrical communication with the tip.

### SUMMARY

A first aspect of the disclosure includes an ultrasonic handpiece assembly comprising: a housing; an electromechanical transducer at least partially disposed within the housing; a tip operatively coupled to the electromechanical transducer; an electrical cord having a console connector, the electrical cord including a first conductor, the first conductor being in electrical communication with the electromechanical transducer; a line connector comprising: a first tubing coupler configured to receive an irrigation line; a second tubing coupler configured to receive an aspiration line; and a receptacle including a first terminal, the receptacle being configured to receive a line extending from a nerve monitor, wherein the first terminal is in electrical communication with the tip.

A second aspect of the disclosure includes a line connector for coupling to an ultrasonic handpiece, the line connector comprising: a first tubing coupler configured to receive an irrigation line; a second tubing coupler configured to receive an aspiration line; and a receptacle including a first terminal, the receptacle being configured to receive a line extending from a nerve monitor.

In some implementations, the line connector is formed from a resilient material.

In some implementations, the housing includes an irrigation conduit, the first terminal being in electrical communication with the irrigation conduit. In some implementations, the line connector comprises a jump conductor arranged to place the first terminal in electrical communication with the irrigation conduit. In some implementations, the line connector comprises a switch, the switch being in electrical communication with the first terminal, and operable to place the first terminal out of electrical communication with the irrigation conduit. In some implementations, the line connector defines a void for receiving a portion of the electrical cord. In some implementations, the line connector includes a base, with the first tubing coupler and the second tubing coupler being formed integrally with the base.

A third aspect of the disclosure includes an ultrasonic handpiece assembly comprising: a housing; an electromechanical transducer at least partially disposed within the housing; a tip operatively coupled to the electromechanical transducer; and an electrical cord having a console connector, the electrical cord including a first conductor, the first conductor being in electrical communication with the electromechanical transducer and the tip, wherein the console connector includes a first terminal in electrical communication with the first conductor, the first terminal for engaging an ultrasonic control console, a second terminal for engaging a source of electrical potential of a nerve monitor, and a jump conductor arranged to place the second terminal in electrical communication with the first conductor.

In some implementations, the console connector includes a third terminal, the third terminal for establishing a connection with a reference probe. In some implementations, the console connector includes a fourth terminal, the fourth terminal for establishing a connection with a stimulator probe, the stimulator probe being separate from the ultrasonic handpiece assembly.

In some implementations, the ultrasonic handpiece assembly further includes a switch, the switch being in electrical communication with the second terminal, and operable to place the second terminal out of communication with the first conductor. In some implementations, the switch is a footswitch. In some implementations, the console connector includes the switch.

A fourth aspect of the disclosure includes an ultrasonic surgical system comprising: an ultrasonic handpiece assembly including: a housing; an electromechanical transducer at least partially disposed within the housing; a tip operatively coupled to the electromechanical transducer; and an electrical cord having a console connector, the electrical cord including a first conductor, the first conductor being in electrical communication with the electromechanical transducer and the tip; wherein the console connector includes a first terminal in electrical communication with the first conductor; and an adapter for connecting the ultrasonic handpiece assembly to a nerve monitor, the adapter comprising a second terminal for engaging an ultrasonic control console, a third terminal for engaging a source of electrical potential of a nerve monitor, and a jump conductor arranged to place the third terminal in electrical communication with the second terminal; wherein, upon connection of the console connector to the adapter, electrical communication is established from the third terminal to the first terminal.

In some implementations, the adapter includes a fourth terminal. In some implementations, the fourth terminal is for establishing a connection with a reference input from the nerve monitor and a sixth terminal for establishing a connection with a reference probe. In some implementations, the fourth terminal is for establishing a connection with a stimulator probe, the stimulator probe being separate from the ultrasonic handpiece assembly.

In some implementations, the ultrasonic surgical system further comprises a switch, the switch being in electrical communication with the third terminal, and operable to place the second terminal out of communication with the first conductor. In some implementations, the switch is a footswitch. In some implementations, the adapter includes the switch.

A fifth aspect of the disclosure includes an ultrasonic surgical system comprising: a control console including a pump and the control console defining a cassette receptacle and a handpiece port for connection to a console connector of an ultrasonic handpiece assembly, the cassette receptacle including a first fluid port and a first terminal; the handpiece port including a second terminal, the first terminal being in electrical communication with the second terminal; a surgical fluid cassette including: a surgical tube; a second fluid port coupled to the surgical tube, the second fluid port being configured to engage the first fluid port to form a fluid connection between the pump and the surgical tube when the surgical fluid cassette is inserted into the cassette receptacle; a third terminal configured to engage the first terminal when the surgical fluid cassette is inserted into the cassette receptacle, such that the first terminal is in electrical communication with the third terminal when the cassette is inserted into the cassette receptacle; and a fourth terminal for being placed in electrical communication with a source of electrical potential of a nerve monitor, the fourth terminal being in electrical communication with the third terminal.

In some implementations, the surgical fluid cassette further comprises a fifth terminal. In some implementations, the fifth terminal is for receiving a reference input from the nerve monitor and a sixth terminal for establishing connection with a reference probe. In some implementations, the fifth terminal is for establishing a connection with a stimulator probe, the stimulator probe being separate from an ultrasonic handpiece assembly.

In some implementations, the ultrasonic surgical system further comprises a ultrasonic handpiece assembly, the ultrasonic handpiece assembly including: a housing, an electromechanical transducer at least partially disposed within the housing, a tip operatively coupled to the electromechanical transducer, and an electrical cord having a console connector, the electrical cord including a first conductor, the first conductor being in electrical communication with the electromechanical transducer and the tip, and the first conductor being in electrical communication with the second terminal.

In some implementations, the ultrasonic surgical system further comprises a switch, the switch being in electrical communication with the third terminal, and operable to place the third terminal out of electrical communication with the first terminal. In some implementations, the switch is a footswitch. In some implementations, the footswitch is connected through the surgical fluid cassette. In some implementations, the surgical fluid cassette includes the switch.

In some implementations, the surgical fluid cassette includes a printed circuit board, the printed circuit board defining the third terminal. In some implementations, the surgical fluid cassette defines an external surface, the printed circuit board defining a portion of the external surface. In some implementations, the printed circuit board is in communication with the fourth terminal. In some implementations, the fourth terminal is a roller pin.

In some implementations, the surgical tube is an aspiration line, and the pump is an aspiration pump. In some implementations, the surgical tube is an irrigation line, and the pump is an irrigation pump.

A sixth aspect of the disclosure includes a surgical system comprising: a control console including a pump and the control console defining a cassette receptacle and a handpiece port for connection to a console connector of a handpiece, the cassette receptacle including a first fluid port and a first terminal; the handpiece port including a second terminal, the first terminal being in electrical communication with the second terminal; a surgical fluid cassette including: a surgical tube; a second fluid port coupled to the surgical tube, the second fluid port being configured to engage the first fluid port to form a fluid connection between the pump and the surgical tube when the cassette is inserted into the cassette receptacle; a third terminal configured to engage the first terminal when the surgical fluid cassette is inserted into the cassette receptacle, such that the first terminal is in electrical communication with the third terminal when the cassette is inserted into the cassette receptacle; and a fourth terminal for being placed in electrical communication with a source of electrical potential of a nerve monitor, the fourth terminal being in electrical communication with the third terminal.

A seventh aspect of the disclosure includes a surgical system comprising: a control console including a pump and the control console defining a cassette receptacle and a handpiece port for connection to a console connector of a handpiece, the cassette receptacle including a first fluid port and a first terminal; the handpiece port including a second terminal, the first terminal being in electrical communication with the second terminal; a surgical fluid cassette including: a surgical tube; a second fluid port coupled to the surgical tube, the second fluid port being configured to engage the first fluid port to form a fluid connection between the pump and the surgical tube when the cassette is inserted into the cassette receptacle; and a third terminal configured to engage the first terminal when the surgical fluid cassette is inserted into the cassette receptacle, such that the first terminal is in electrical communication with the third terminal when the cassette is inserted into the cassette receptacle.

These and other configurations, features, and advantages of the present disclosure will be apparent to those skilled in the art. The present disclosure is not intended to be limited to or by these configurations, embodiments, features, and/or advantages.

### BRIEF DESCRIPTION OF THE DRAWINGS

Referring now to the drawings, exemplary illustrations are shown in detail. Although the drawings represent schematic embodiments, the drawings are not necessarily to scale and certain features may be exaggerated to better illustrate and explain an innovative aspect of an illustrative embodiment. Further, the exemplary illustrations described herein are not intended to be exhaustive or otherwise limiting or restricting to the precise form and configuration shown in the drawings and disclosed in the following detailed description.

Advantages of the present invention will be readily appreciated as the same becomes better understood by reference to the following detailed description when considered in connection with the accompanying drawings wherein:

FIG. 1 is a side view of an ultrasonic surgical handpiece assembly, including an ultrasonic handpiece, an irrigation sleeve, an ultrasonic tip, and a control console.

FIG. 2 is a top view of the ultrasonic surgical handpiece assembly of FIG. 1.

FIG. 3 is a sectional view of the ultrasonic surgical handpiece assembly of FIG. 1.

FIG. 4 is a zoomed (enhanced) sectional view of a rear housing portion of the ultrasonic handpiece of FIG. 3.

FIG. 5 is a perspective view of a front housing portion of the ultrasonic handpiece of FIG. 1.

FIG. 6 is an exploded view of the front housing portion of FIG. 4.

FIG. 7 is a front view of the ultrasonic handpiece of FIG. 6.

FIG. 8 is a partially exploded view of the ultrasonic surgical handpiece assembly of FIG. 1, illustrating an example configuration of an interface between the front housing portion of the ultrasonic handpiece and the irrigation sleeve.

FIG. 9 is a zoomed (enhanced) sectional view of the front housing portion of the ultrasonic handpiece coupled to the irrigation sleeve.

FIG. 10 is a perspective view of the irrigation sleeve and the ultrasonic tip of the ultrasonic surgical handpiece assembly FIG. 1, for use with the ultrasonic handpiece.

FIG. 11A is a bottom view of the irrigation sleeve and the ultrasonic tip of FIG. 10.

FIG. 11B is a sectional view of the irrigation sleeve and the ultrasonic tip of FIG. 10.

FIG. 12 is a perspective view of an example configuration of a tubing connector for use with the ultrasonic surgical handpiece assembly FIG. 1, including the ultrasonic handpiece shown in phantom.

FIG. 13 is a first perspective view of the tubing connector of FIG. 12.

FIG. 14 is a second perspective view of the tubing connector of FIG. 12.

FIG. 15 is a third perspective view of the tubing connector of FIG. 12.

FIG. 16 is a cut-away sectional view of the tubing connector of FIG. 12 coupled to an ultrasonic handpiece with power, aspiration, and irrigation lines extending therefrom.

FIG. 17 is a view of an ultrasonic surgical handpiece in proximity to one or more nerve locations.

FIG. 18 is an electrical schematic for the ultrasonic surgical system.

FIG. 19 is a perspective view of an ultrasonic surgical system with an adapter.

FIG. 20 is an exploded view of the components of the system of FIG. 19.

FIG. 21 is a schematic diagram for the system of FIG. 19.

FIGS. 22A and 22B are exploded views of the adaptor of FIG. 19.

FIG. 23 is a schematic diagram of the system of FIG. 19.

FIG. 24 is a perspective view of another potential implementation of an ultrasonic surgical system.

FIG. 25 is a schematic view of a surgical fluid cassette.

FIG. 26 is a schematic view of the ultrasonic surgical system of FIG. 24.

FIGS. 27A and 27B are a perspective views of the internal components of the surgical fluid cassette and surgical console from FIG. 24.

FIG. 28 illustrates potential conductive pathways within the surgical fluid cassette of FIG. 24.

FIG. 29 is a perspective view of a line connector of another potential implementation of an ultrasonic surgical system.

FIGS. 30A and 30B are perspective views of the ultrasonic surgical system of FIG. 29

FIG. 31 is a schematic view of the ultrasonic surgical system of FIG. 29.

FIG. 32 is a cut-away sectional view of the line connector and an ultrasonic surgical handpiece of the ultrasonic surgical system of FIG. 29.

### DETAILED DESCRIPTION

As medical professionals strive for reducing the size of the incisions and the amount of recovery time required following invasive medical procedures, the size of medical instruments used in various medical procedures have become smaller. Many of the medical instruments utilized in performing the various medical procedures may include the use of a cutting accessory, such as ultrasonic handpieces, high-speed drills, rotating burs, open-window shavers, and the like. Many of these cutting accessories may require the use of irrigation or aspiration (i.e., suction) to reduce heat and/or remove debris at the surgical site. Similarly, irrigation may be utilized to lubricate a cutting accessory.

One example of a surgical instrument that may utilize irrigation and aspiration systems is an ultrasonic handpiece. Generally, one or more lines may be coupled to the ultrasonic handpiece to supply irrigation and suction. The ultrasonic handpiece may further comprise a sleeve comprising one or more lumens that may be utilized to direct fluid from an irrigation source toward the surgical site and the cutting accessory, i.e., an ultrasonic tip. Irrigation and/or aspiration lines have typically been coupled to the exterior of the ultrasonic handpiece to be coupled to the sleeve. The sleeve and/or cutting accessory may be disposable, resulting in the sleeve and/or cutting accessory only temporarily being attached to the ultrasonic handpiece. The introduction of additional elements, such as the irrigation and/or aspiration lines to the exterior of the handpiece can result in increasing the size, profile, and/or general bulkiness of the ultrasonic handpiece. This may obstruct the medical professional's view and/or distract the medical professional during operation of the medical instrument during the medical procedure. The positioning of the irrigation and/or aspiration lines relative to the ultrasonic handpiece may similarly affect the ergonomics of the ultrasonic handpiece, inhibiting the medical professional's ability to manipulate the ultrasonic handpiece.

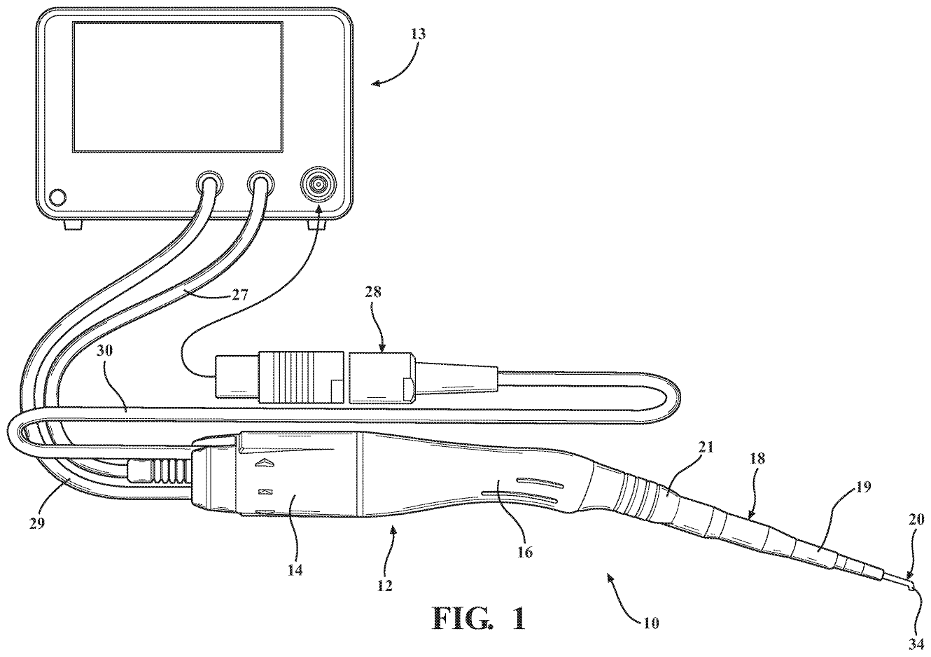

Therefore, an ultrasonic surgical handpiece assembly 10 may be configured to comprise an ultrasonic handpiece 12 including internal irrigation and aspiration lumens to reduce the size of the handpiece and improve ergonomics, such as the ultrasonic surgical handpiece assembly 10 illustrated in FIGS. 1-3.

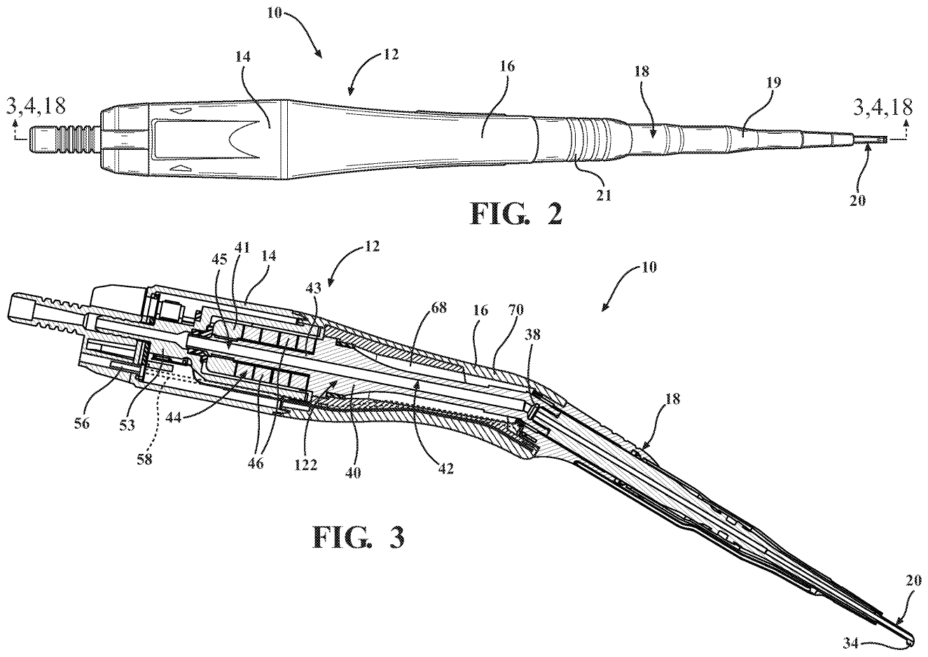

Referring to FIGS. 1 and 2, an example configuration of an ultrasonic surgical handpiece assembly 10 is illustrated. The ultrasonic surgical handpiece assembly 10 may comprise an ultrasonic handpiece 12 comprising a proximal end 14 and distal end 16. The ultrasonic surgical handpiece assembly 10 may further comprise sleeve 18 and an ultrasonic tip 20 that may be coupled to the distal end of the ultrasonic handpiece 12. The sleeve 18 may be configured to provide irrigation to the ultrasonic tip 20 and/or the surgical site. It is further contemplated that the sleeve 18 may also be configured to provide aspiration to the ultrasonic tip 20. The ultrasonic tip 20 may comprise a cutting feature 34 that is configured to cut, shape, and/or remove biological tissue.

The ultrasonic tip 20 may have various features, as described in U.S. Pat. Nos. 6,497,715; 6,955,680; and 6,984,220; which are hereby incorporated herein by reference in their entirety.

The ultrasonic surgical handpiece assembly 10 may also comprise a cable 30 or other power cord comprising a power connector 28 or adapter configured to couple the ultrasonic surgical handpiece assembly 10 to a power supply, such as a control console 13 configured to regulate the various aspects of the ultrasonic handpiece 12. For example, the console 13 may be configured to regulate the power and the signal supplied to the ultrasonic handpiece 12. The console 13 may also be configured to regulate the irrigation and/or aspiration functions of the ultrasonic handpiece 12 to optimize performance of the ultrasonic surgical handpiece assembly 10.

Referring to FIG. 3, a sectional view of the ultrasonic surgical handpiece assembly 10 of FIG. 1 is provided. The ultrasonic handpiece 12 may comprise a proximal housing portion 14 and a distal housing portion 16, each of which may be configured to define a void. The proximal housing portion 14 and the distal housing portion 16 may be configured as two separate components and may be coupled together by a laser weld or similar coupling process. Alternatively, proximal housing portion 14 and the distal housing portion 16 may including corresponding coupling features configured to couple the proximal housing portion 14 and the distal housing portion 16 together. It is also contemplated that the proximal housing portion 14 and the distal housing portion 16 may be configured as a single unitary component.

A transducer 44 may be disposed in the void defined by the ultrasonic handpiece 12. The transducer 44 may comprise a plurality of driver elements 46, such as piezoelectric crystals arranged in a stacked configuration. The piezoelectric crystals 46 may expand and contract based on the varied application of electricity. The transducer 44 may comprise a tube 43 that defines a lumen 45 that extends from the proximal end to the distal end of the transducer 44 to create a fluid passageway through the transducer 44. The tube 43 may take the form of a post. The tube 43 may extend through the collinear longitudinal axes of the driver elements 46. A proximal end mass 41 may be located adjacent to the proximal face of the most proximally located driver element 46.

It is further contemplated that the transducer 44 may alternatively include a plurality of magnetostrictive elements.

A horn 40 may be at least partially disposed within the void defined by ultrasonic handpiece 12. The horn 40 may be coupled to the distal end of the transducer 44. The horn 40 may be constructed from a rigid steel alloy, titanium or similar material. In operation, as the transducer 44 expands and contracts, the horn 40 will oscillate. The horn 40 may be removably coupled to the transducer 44. For example, the proximal end of the horn 40 may comprise a threaded male coupler and the distal end of the transducer 44 may comprise a corresponding female threaded coupler. Alternatively, the transducer 44 and the horn 40 may be permanently coupled via a weld, adhesive, or similar bonding process. The horn 40 may be configured to define a second conduit 42 that is in fluid communication with the lumen 45 defined by the tube 43 of the transducer 44. Collectively, the lumen 45 through the transducer 44 and the second conduit 42 through the horn 40 form a portion of a continuous fluid passageway that extends from the distal end of the ultrasonic handpiece 12 to the proximal end of the ultrasonic handpiece 12. The ultrasonic surgical handpiece assembly 10 is constructed so that the driver elements 46 are compressed between the proximal end mass 41 and the horn 40.

The fluid passageway through the ultrasonic handpiece 12 may be utilized to provide irrigation fluid and/or a vacuum through the ultrasonic handpiece 12. The distal end of the horn 40 may further comprise a threaded coupler 38 that is configured to removably couple the ultrasonic tip 20 to ultrasonic handpiece 12 via the horn 40. While the threaded coupler 38 on the distal end of the horn 40 may comprise threads configured to engage corresponding threads on the ultrasonic tip 20, it is further contemplated that other coupling methods may be utilized. For example, the distal end of the horn 40 may comprise features that allow snap fit engagement with the ultrasonic tip 20.

The control console 13 of the ultrasonic surgical handpiece assembly 10 may be configured to source drive signals over the cable 30 to which the ultrasonic handpiece 12 is connected. In many but not all versions of ultrasonic surgical handpiece assembly 10, the ultrasonic handpiece 12 and cable 30 are assembled as a single unit. The drive signals are applied to the piezoelectric crystals 46. At any given instant, the same drive signal is applied to each of plurality of piezoelectric crystals 46. The application of the drive signals causes the piezoelectric crystals 46 to simultaneously and cyclically expand and contract. The horn 40 may be configured to amplify this movement. Consequently, the distal end of the horn 40 and, by extension, the ultrasonic tip 20, when moving from the fully contracted position to the fully extended position typically moves a maximum of 1000 microns and more often 500 microns or less. Some ultrasonic tips 20 are further designed to so that the longitudinal extension/retraction of the ultrasonic tip 20 may also induces a torsional movement in the cutting feature 34. When ultrasonic handpiece 12 is actuated to cause the cyclic movement of the ultrasonic tip 20, the cutting feature 34 is considered to be vibrating.

The control console 13 may also include a vacuum pump and processor/controller, and an irrigation pump and a processor/controller. The vacuum pump may be coupled to the ultrasonic surgical handpiece assembly 10 via aspiration line 27. The irrigation pump may be coupled to the ultrasonic handpiece assembly 10 via irrigation line 29.

The control console 13 may have any of the features described in U.S. Patent Publication No. 2017/0071621 and 2018/0056328, which are hereby incorporated by reference in their entirety.

The ultrasonic handpiece 12 may also comprise a tube 58 that is at least partially disposed within the ultrasonic handpiece 12 and configured to define a lumen 59 through the ultrasonic handpiece 12 for irrigation and/or suction. The tube 58 defines an additional lumen 59 through the ultrasonic handpiece 12 to the passageway defined by the lumen 45 of the transducer 44 and the second conduit 42 of the horn 40. For example, the tube 58 may define an irrigation lumen 59 through the ultrasonic handpiece 12 and the lumen 45 through the transducer 44 and the second conduit 42 through horn 40 may define an aspiration lumen 45 through the ultrasonic handpiece 12. Alternatively, the tube 58 may define an aspiration passageway 59 through the ultrasonic handpiece 12 and the lumen 45 through the transducer 44 and the second conduit 42 through the horn 40 may define an irrigation passageway through the ultrasonic handpiece 12.

As described above, the irrigation and/or the aspiration passageways are disposed mostly within the ultrasonic handpiece 12 to provide ergonomic advantages by eliminating the one or more tubes typically attached to the exterior of the ultrasonic handpieces. It should be understood that while portions of the lines that define the aspiration passageway and/or the irrigation passageway from the proximal end of the ultrasonic surgical handpiece assembly 10 to the distal end of the passageway may be exposed, i.e., visible to a user during operation, there are is no need for users to attach any irrigation lines or aspiration lines to the sides of the ultrasonic surgical handpiece assembly 10 during use. Instead, the irrigation lines 29 and aspiration lines 27, in certain embodiments, solely attach to the proximal end, even the rear face of the proximal end of the ultrasonic handpiece 12. In addition, in certain embodiments, during set-up of the ultrasonic surgical handpiece assembly 10, all of the aspiration and irrigation connections that the sleeve includes are sourced directly from the ultrasonic handpiece 12. In other words, there are no irrigation or aspiration lines that couple to a side of the sleeve 18. Thus, the ultrasonic handpiece 12 may include connections for both irrigation and aspiration.

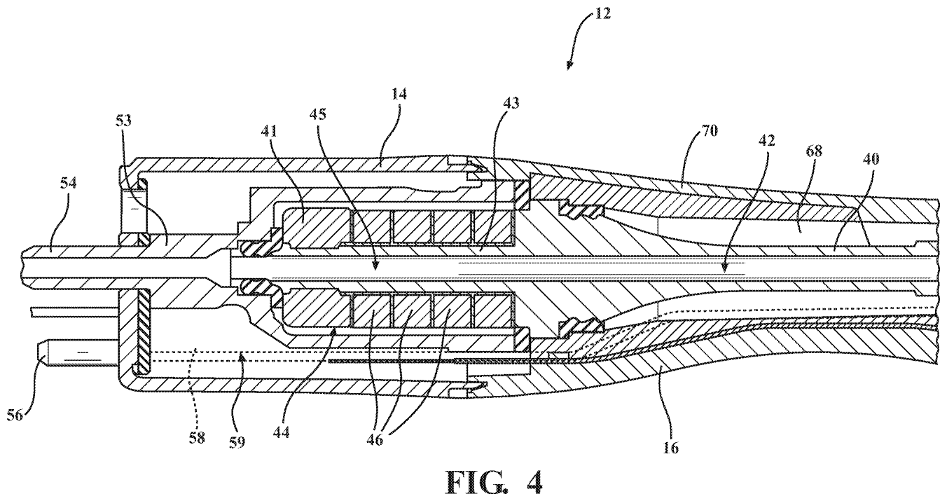

Referring to FIG. 4, a sectional view of a portion of the ultrasonic handpiece 12 is illustrated. As described above, the transducer 44 may comprise a distal end and a proximal end disposed within the void of ultrasonic handpiece 12. The transducer 44 may be configured to expand and contract along the longitudinal axis of the transducer 44. The lumen 45 through the transducer 44 comprises a distal portion and a proximal portion. The horn 40 may also be at least partially disposed within the ultrasonic handpiece 12.

The ultrasonic handpiece 12 may further comprise a barrier member 49 positioned within the void defined by the ultrasonic handpiece 12 and configured to define a cavity. The transducer 44 may be encased within the barrier member 49, such that the barrier member 49 may be configured to assist in mounting and isolating the transducer 44 within the ultrasonic handpiece 12. The barrier member 49 may not occupy the entirety of the void defined within the ultrasonic handpiece 12. Therefore, the ultrasonic handpiece 12 may further comprise a potting element 47 or material that is disposed within void defined by the ultrasonic handpiece 12. The potting element 47 may be disposed within the void defined by the ultrasonic handpiece 12 and exterior to the barrier member 49, such that the potting element 47 may occupy or fill the portion of the void within the handpiece 12 that is not occupied by the barrier member 49 or occluded by the barrier member 49. The potting element 47 may be configured to fix the position of the barrier member 49 within the void defined by the ultrasonic handpiece 12. The potting element 47 may also function as an insulator or dampener configured to prevent the transfer of thermal energy (i.e., heat) and mechanical energy (i.e., vibration) from the transducer 44 to the user's hand.

The barrier member 49 may further define a channel 53 extending in a generally proximal direction from the proximal end of the barrier member 49. The channel 53 forms a passageway between the proximal end of the ultrasonic handpiece 12 and the tube 43 that defines the lumen 45 through the transducer 44. The channel 53 may further comprise a coupling portion 54, such as a hose barb or similar fitting configured to create a friction fit, that extends proximally from the proximal end of the ultrasonic handpiece 12 and is configured to couple the channel 53 to the aspiration line 27 of the control console 13.

The transducer 44 may be partially mounted in the cavity defined by the barrier member 49 by a rear seal 48 positioned between said proximal end of the transducer 44 and an interior surface of the barrier member 49. The rear seal 48 may be configured to abut said proximal end of the transducer 44 and define a first aperture that is in fluid communication with the lumen 45 through the transducer 44. The rear seal 48 is positioned to help prevent moisture ingress to the volume that surrounds the transducer 44, in between the barrier member 49 and the exterior of the transducer 44. The rear seal 48 also functions to prevent moisture from entering between the exterior surface of the tube 43 and the interior surface of the transducer 44. The rear seal 48 may be formed from an elastomeric material that is resistant to heat and vibration degradation, including those materials that can withstand temperatures of an autoclave process.

The ultrasonic handpiece 12 may further comprise a front seal 52 disposed radially about an exterior surface of the horn 40 to prevent moisture ingress between an interior surface of the distal housing portion 16 and the horn 40. This is because the exterior surface of the horn 40 will be exposed to liquid during operation of the ultrasonic handpiece 12. The front seal 52 may further comprise a plurality of protrusions or bumps configured to facilitate engagement between the horn 40 and the distal housing portion 16 and prevent ingress of moisture into the void between the horn 40 and the distal housing portion 16 of the ultrasonic handpiece 12.

The ultrasonic handpiece 12 may further comprise a potting seal 50 positioned between and engaging both a distal end of the barrier member 49 and the proximal end of the horn 40. The potting seal 50 may also be configured to contact an interior surface of distal housing portion 16 of the ultrasonic handpiece 12 that is adjacent to the barrier member 49 and the proximal end of the horn 40. The potting seal 50 may be configured to abut the distal end of the barrier member 49 and define a second aperture for receiving the coupling feature of the horn 40. The potting seal 50 may be configured to prevent the potting element 47 from entering the barrier member 49 and contacting the transducer 44 during the potting process. Once the potting element 47 is in place, the potting element 47 in conjunction with the potting seal 50 helps to prevent moisture from entering between the barrier member 49 and the horn 40.

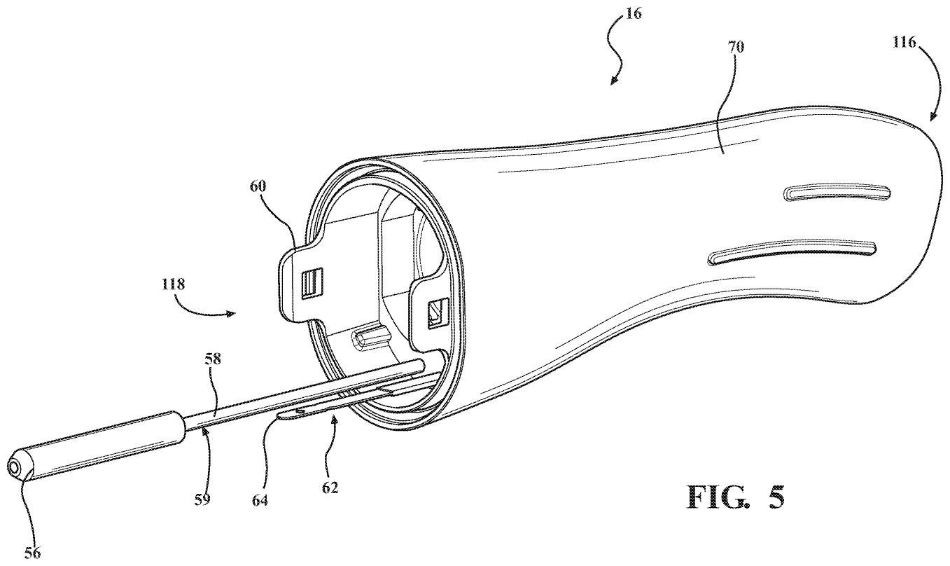

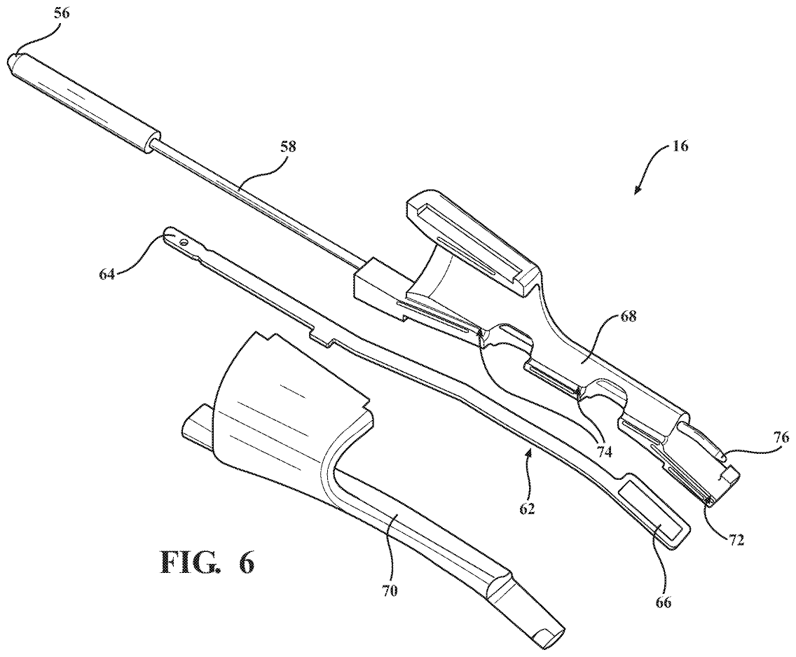

Referring to FIGS. 5 and 6, an exemplary configuration of the distal housing portion 16 is illustrated. The distal housing portion 16 may comprise a coupling feature 60 configured to removably couple the distal housing portion 16 to a distal portion of the barrier member 49 that is disposed within the proximal housing portion 14. The coupling feature 60 may comprise a pair of tabs configured to create a snap and/or friction fit with a corresponding coupling feature of the barrier member 49. However, alternative coupling features are also contemplated, such as hooks or protrusions. The coupling feature 60 may assist in mounting and/or positioning the barrier member 49 within the void defined by the distal housing portion 16 and the proximal housing portion 14.

The distal housing portion 16 of the ultrasonic handpiece 12 may further comprise a flex circuit 62 that may be molded into the distal housing portion 16. The flex circuit 62 may be constructed from an electrically conductive material configured to transmit electrical signals between a distal region and proximal region of the flex circuit 62. The flex circuit 62 may comprise an attachment portion 64 positioned at the proximal region of the flex circuit 62 and configured to couple to a processor disposed within the ultrasonic handpiece 12 that is configured to communicate with the control console 13. The flex circuit 62 may also comprise a transceiver 66 positioned at the distal region of the flex circuit 62.

Also illustrated in FIG. 5 is the tube 58 defining the lumen 59 described above, that is at least partially disposed within the ultrasonic handpiece 12. The tube 58 may be at least partially disposed within the distal housing portion 16 and the proximal housing portion 14. The tube 58 may further comprise a coupling feature 56, such as a hose barb or similar fitting configured to create a friction fit, positioned at proximal end of the tube 58 and configured to couple to the tube 58 to an irrigation line 29 that is routed from the control console 13 to the ultrasonic surgical handpiece assembly 10. The irrigation line 29 from the console 13 may be coupled to the ultrasonic handpiece 12 via the hose barb 56 to facilitate the flow of irrigation fluid through tube 58 within the ultrasonic handpiece 12 to the sleeve 18 and/or ultrasonic tip 20. In the configuration described above, the irrigation fluid may flow from the proximal end to the distal end of the ultrasonic handpiece 12 within the ultrasonic handpiece 12 or sleeve 18 eliminating the need for bulky irrigation lines coupled to the side of the ultrasonic handpiece 12 or the side of the sleeve 18. The routing of irrigation lines through the handpiece 12 enables this configuration. Thus, neither the sleeve, nor the ultrasonic handpiece includes an irrigation coupling part along the sides. Alternatively, in other configurations, the tube 58 may be coupled to an aspiration line 27 via the coupling feature 56. The aspiration line 27 may be utilized to draw a vacuum through the tube 58. The tube 58 may be in communication with the sleeve 18 and/or ultrasonic tip 20 to remove biological material and/or fluid from the surgical site.

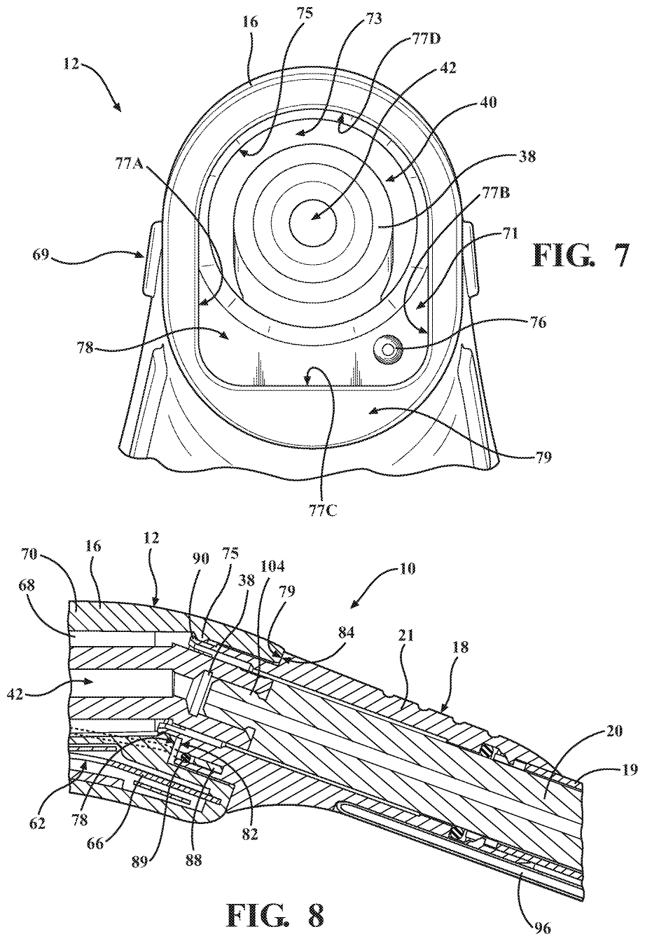

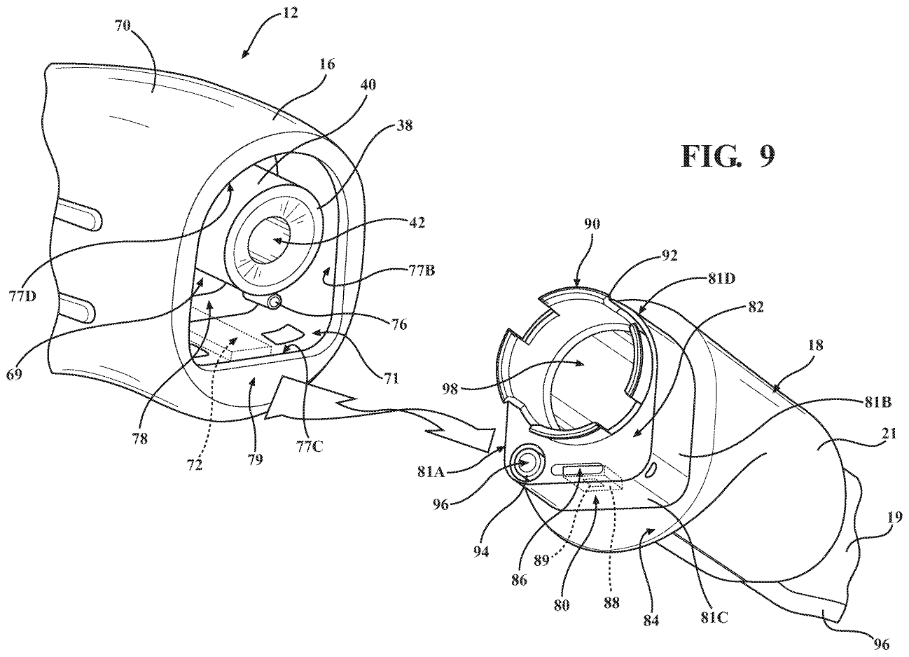

Referring to FIGS. 7-9, an exemplary configuration of the interface between the distal housing portion 16 and the sleeve 18 and/or ultrasonic tip 20 is illustrated. FIG. 7 illustrates a front view of an example configuration of the distal end of the distal housing portion 16 described above. This may also be referred to as the distal end of the ultrasonic handpiece 12. The distal end of the ultrasonic handpiece 12 may comprise an attachment region 69 configured to serve as the interface between the ultrasonic handpiece 12 and the sleeve 18 and the ultrasonic tip 20. The attachment region 69 may comprise a recess 71 defined by a proximal surface 78 and a distal surface 79 that are connected by a plurality of walls 77 extending perpendicular to the proximal surface 78 and the distal surface 79. The proximal surface 78 may comprise an aperture 73 including a coupling feature 75 at the perimeter of the aperture 73 (See FIG. 9). The coupling feature 75 may comprise a tab, lip, or similar snap fit coupling mechanism.

The distal end of the horn 40 may be configured to partially extend through the aperture 73 defined in the proximal surface 78, wherein at least a portion of the horn 40 and the threaded coupler 38 are disposed within the recess 71 at the distal end of the ultrasonic handpiece 12. The second conduit 42 that extends through the horn 40 may similarly open to the recess 71.

The proximal surface 78 may further comprise an irrigation coupler 76 positioned at the distal end of the tube 58 defining the lumen 59 through the ultrasonic handpiece 12. The irrigation coupler 76 may comprise a fitting, hose barb, or similar coupling member for coupling the distal end of the tube 58 to a corresponding irrigation line 29 of the sleeve 18. The irrigation coupler 76 may be configured to be in fluid communication with the lumen 59 disposed within the ultrasonic handpiece 12 to provide irrigation fluid to the sleeve 18. The irrigation coupler 76 may serve as an inlet or an outlet to transport material and/or fluid between the ultrasonic handpiece 12 and sleeve 18.

Referring to FIG. 8, a partially exploded view of the interface between the distal end of the ultrasonic handpiece 12 and the sleeve 18 is illustrated. The sleeve 18 may comprise a hub 21 positioned at a proximal end of the sleeve 18 and a sleeve body 19 that extends distally from the hub 21. The sleeve 18 may comprise a lumen 98 defined by the hub 21 and sleeve body 19 that extends the length of sleeve 18 and is configured to surround at least a portion of the ultrasonic tip 20 when coupled to the ultrasonic handpiece 12. The hub 21 may comprise a protrusion 80 that extends proximally from a distal surface 84 of the hub 21 and terminates at a proximal surface 82. The proximal surface 82 of the protrusion 80 may be oriented to be generally parallel to the distal surface 84. The shape of the protrusion 80 may be defined by a plurality of external circumferential surfaces 81 or wall members that extend between the distal surface 84 and the proximal surface 82 and are configured to define the outer perimeter of the protrusion 80. The external circumferential surfaces 81 may be oriented to be generally perpendicular to the proximal surface 82 and/or the distal surface 84.

The hub 21 may further comprise a fitting 94 extending from the proximal surface 82 of the protrusion 80. The fitting 94 may be configured to couple to the corresponding fitting 76 of the ultrasonic handpiece 12. The fitting 94 of the sleeve 18 and corresponding fitting 76 of the ultrasonic handpiece 12 may be configured to facilitate the exchange of fluid between the sleeve 18 and the ultrasonic handpiece 12. In particular, the fitting 76 is sized to fit within the fitting 94 when the sleeve 18 is coupled to the ultrasonic handpiece 12. For example, irrigation fluid from the ultrasonic handpiece 12 may be dispersed to the sleeve 18 through the connection of the fitting 94 to the fitting 76. This eliminates the need to connect the sleeve to an irrigation line that is separate from the ultrasonic handpiece 12, e.g., such as an irrigation line that connects to a side of the sleeve 18. Alternatively, a vacuum may be applied to the ultrasonic handpiece 12 configured to aspirate material from the sleeve 18 through the fittings 94 and 76 when coupled in an alternative configuration.

The sleeve 18 may further comprise a conduit 96 formed in the sleeve body 19 that runs adjacent to the lumen 98 of the sleeve 18. The fitting 94 may be coupled to the proximal end of the conduit 96. The conduit 96 may be configured to channel irrigation fluid through the sleeve 18 separate from the lumen 98 of the sleeve 18. The conduit 96 may be routed in various ways through the sleeve 18.

The hub 21 may further comprise a cavity 86 in the protrusion 80 that that includes an opening in the proximal surface 82 of the protrusion 80. The cavity 86 may be configured to at least partially enclose a tag 88, antenna, transceiver, or similar wireless communication device that is configured to communicate with the transceiver 66 disposed in the distal housing portion 16 of the ultrasonic handpiece 12.

As illustrated in FIG. 9, the tag 88 may be secured in the cavity 86 by a pin 89. The pin 89 may comprise a plastic or alloy material and be configured to create a friction fit within the opening of the cavity 86 to secure the RFID tag 88 within the cavity 86 and protect it from being damaged.

The protrusion 80 may be configured in a complementary shape to the one defined by the recess 71 at the distal end of the ultrasonic handpiece 12, wherein the protrusion 80 is configured to be disposed within the recess 71 when the sleeve 18 is coupled to the ultrasonic handpiece 12. Furthermore, the shape of the recess 71 and the protrusion 80 may be configured to align the sleeve 18 relative to the ultrasonic handpiece 12, as well as prevent rotational movement of the sleeve 18 relative to the ultrasonic handpiece 12. For example, as illustrated in FIG. 8, the recess 71 of the ultrasonic handpiece 12 comprises a plurality of walls 77 that define the perimeter of the recess 71. In the example configuration of the recess 71 illustrated in FIGS. 7-8, the walls 77A, 77B, 77C, and 77D define a doghouse-like shape. The plurality of external circumferential surfaces 81A, 81B, 81C, and 81D that define the outer perimeter of the protrusion 80 may then be configured to define a doghouse-like shape that corresponds to the shape of the recess 71.

The shape of the protrusion 80 and the recess 71 may further be configured to orient the tag 88 that is disposed in the cavity 86 of the hub 21 relative to the transceiver 66 disposed in the distal housing portion 16 of the ultrasonic handpiece 12. Utilizing the shape of the protrusion 80 and the recess 71 to orient the tag 88 relative to the transceiver 66 can improve the reliable establishment of communication between the tag 88 relative and the transceiver 66.

The hub 21 may further comprise a plurality of retention fingers 90 that extend distally from the proximal surface 82 of the protrusion 80. The plurality of retention fingers 90 may be spaced about the perimeter of the lumen 98 defined in the proximal surface 82. The plurality of retention fingers 90 may further comprise a tab 92, bump, or protrusion configured to engage the coupling feature 75 at the perimeter of the aperture 73 in the proximal surface 78 of the recess 71 in the ultrasonic handpiece 12. The tab 92 of each of the plurality of retention fingers 90 may engage the coupling feature 75 of the recess 71 to create a snap-fit and/or friction fit to removably couple the sleeve 18 to the ultrasonic handpiece 12.

Referring to FIG. 9, a sectional view of an example configuration of the interface between the distal housing portion 16 of the ultrasonic handpiece 12 and the sleeve 18 and ultrasonic tip 20 is illustrated. As illustrated in FIG. 9, the distal surface 79 of the distal housing portion 16 may abut the distal surface 84 of the protrusion 80 when the sleeve 18 is coupled to the ultrasonic handpiece 12. Similarly, the proximal surface 78 of the distal housing portion 16 may abut the proximal surface 82 of the protrusion 80 when the protrusion 80 is disposed within the recess 71 to couple the sleeve 18 to the ultrasonic handpiece 12.

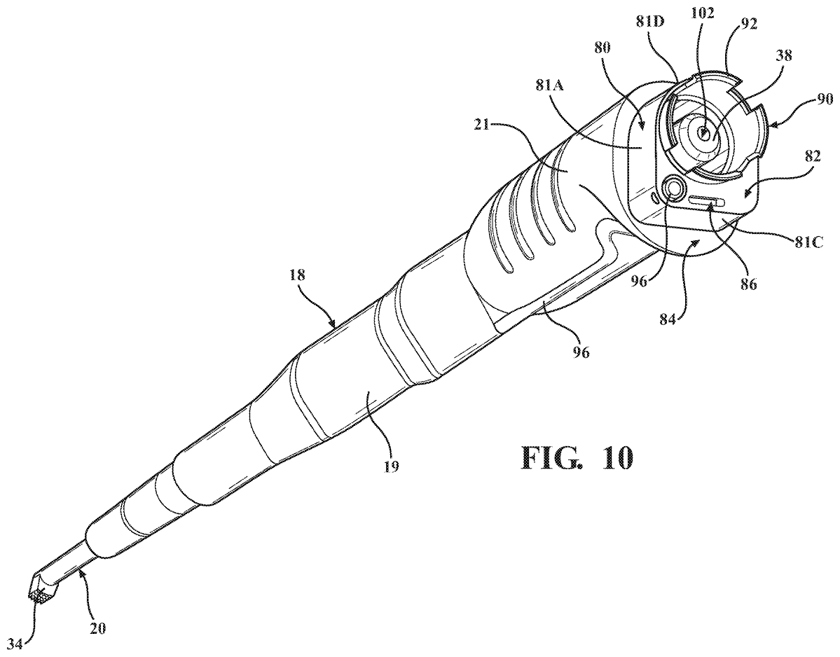

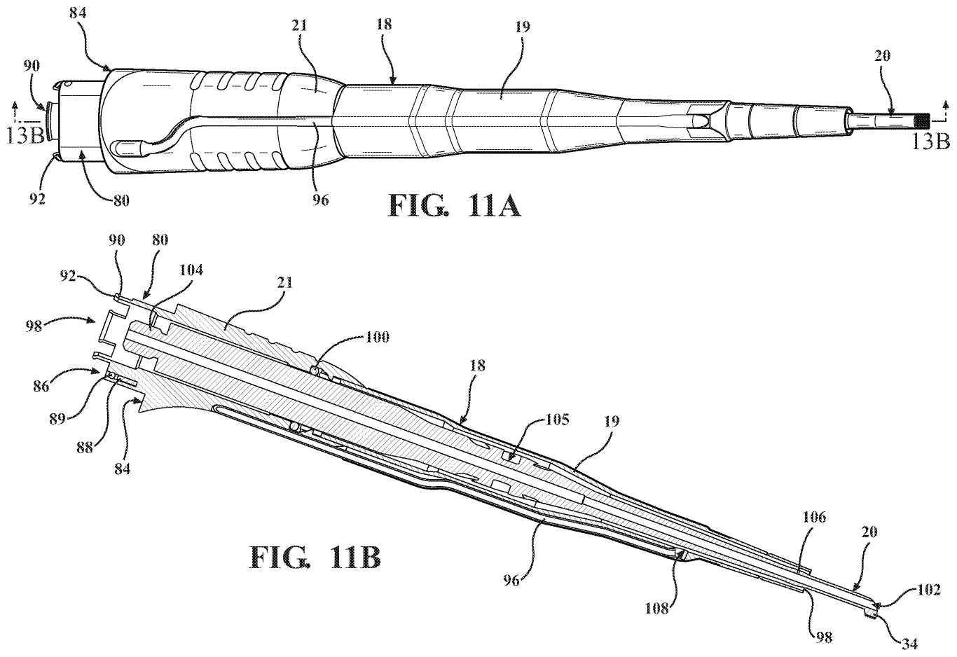

Referring to FIGS. 10, 11A, and 11B, an example configuration of the ultrasonic tip 20 disposed within the sleeve 18 is illustrated. Referring to FIG. 11B, a sectional view of the ultrasonic tip 20 disposed within the sleeve 18 is illustrated. As described above, the sleeve 18 may comprise the conduit 96 formed in the sleeve body 19 and runs adjacent to the lumen 98 of the sleeve 18. The distal end of the conduit 96 may terminate at an aperture 108 or nozzle positioned on an interior surface of the lumen 98. The aperture 108 may configured to direct fluid radially inward toward the center of the lumen 98. The fluid may then be applied to an exterior surface of the ultrasonic tip 20 and/or directed toward the distal end of the sleeve 18, wherein the fluid may exit the lumen 98 and be applied to the surgical site.

The sleeve 18 may further comprise a seal 100, such as O-ring, disposed within the lumen 98 and configured to contact a portion of the ultrasonic tip 20 when the sleeve 18 is coupled to the ultrasonic handpiece 12 and the ultrasonic tip 20 is coupled to the ultrasonic handpiece 12.

The ultrasonic tip 20 may comprise a distal end and a proximal end. The ultrasonic tip 20 may include a cutting feature 34 at the distal end of ultrasonic tip 20 and a coupling feature 104, positioned at the proximate end. The coupling feature 104 may be configured to engage the threaded coupler 38 of the horn 40 to removably couple the ultrasonic tip 20 to the ultrasonic handpiece 12.

The ultrasonic tip 20 may be configured to define a lumen 102 that extends from the proximal end to the distal end of the ultrasonic tip 20. The lumen 102 may be configured to be in fluid communication with the fluid passageway through the ultrasonic handpiece 12 that is defined by the conduit 42 of the horn 40 and the lumen 45 of the transducer 44 when the ultrasonic tip 20 is coupled to the ultrasonic handpiece 12. The lumen 102 may be configured to provide irrigation and/or suction at the surgical site based on the system that is coupled to the fluid passageway through the ultrasonic handpiece 12 that is defined by the conduit 42 of the horn 40 and the lumen 45 of the transducer 44. For example, if an irrigation system is coupled to the fluid passageway through the ultrasonic handpiece 12 that is defined by the conduit 42 of the horn 40 and the lumen 45 of the transducer 44, the lumen 102 may be configured to provide irrigation fluid to the surgical site proximate the cutting feature 34. Alternatively, if an aspiration system is coupled to the fluid passageway through the ultrasonic handpiece 12 that is defined by the conduit 42 of the horn 40 and the lumen 45 of the transducer 44, the lumen 102 may configured to remove fluid and/or material from the surgical site proximate the cutting feature 34.

The ultrasonic tip 20 may also comprise an aperture 106 that is configured to be in fluid communication with the lumen 102. The aperture 106 may be positioned between the proximal end and distal end of the ultrasonic tip 20 such that the aperture 106 is distal of the seal 100 of the sleeve 18 when the ultrasonic tip 20 is disposed within the lumen 98 of the sleeve 18. Furthermore, the aperture 106 may positioned on the ultrasonic tip 20 such that the aperture 106 is surrounded by the sleeve 18 when the ultrasonic tip 20 is disposed within the lumen 98 of the sleeve 18.

The ultrasonic tip 20 may further comprise a resonator 105 positioned between the proximal end and the distal end of the ultrasonic tip 20. The resonator 105 may be configured to translate the longitudinal vibration transmitted from the transducer 44 to the ultrasonic tip 20 via the mechanical connection created by the horn 40 into longitudinal and torsional motion of the ultrasonic tip 20 distal to the resonator 105.

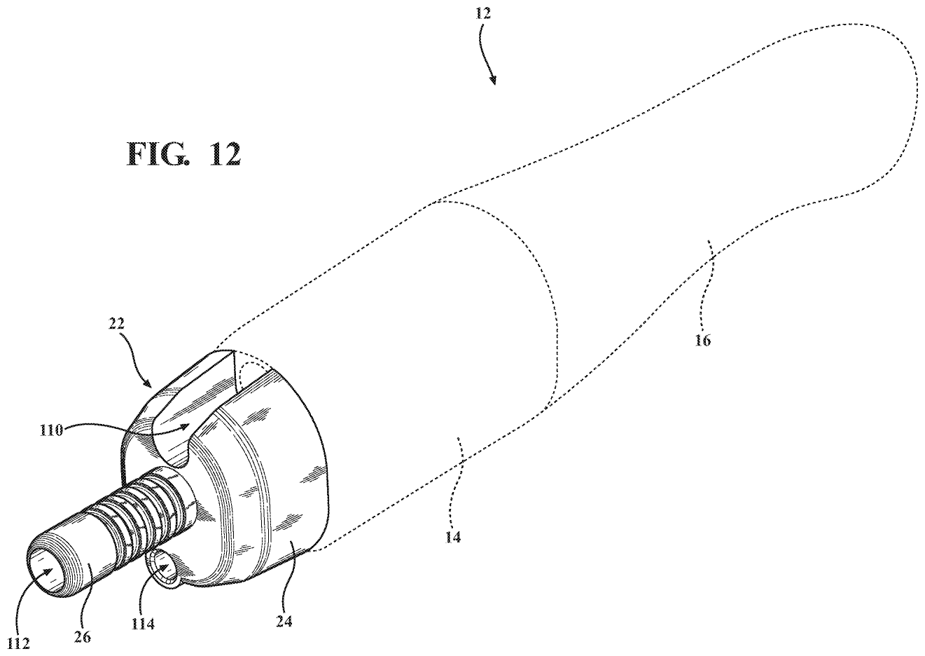

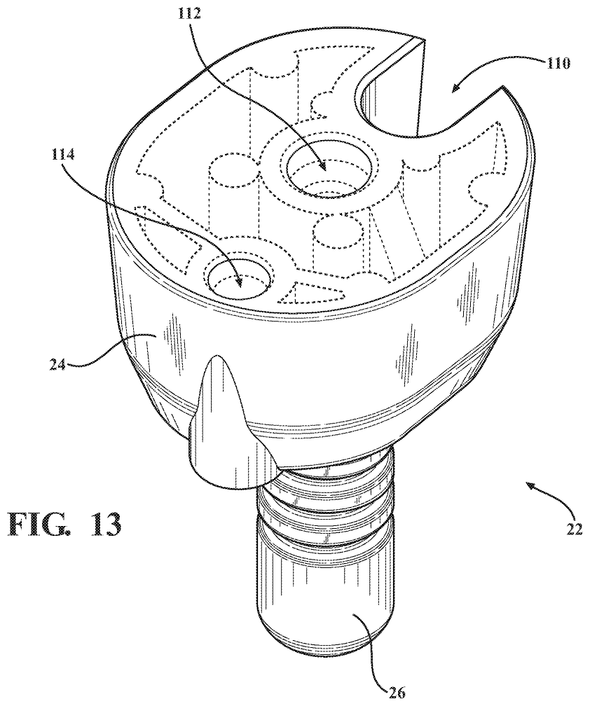

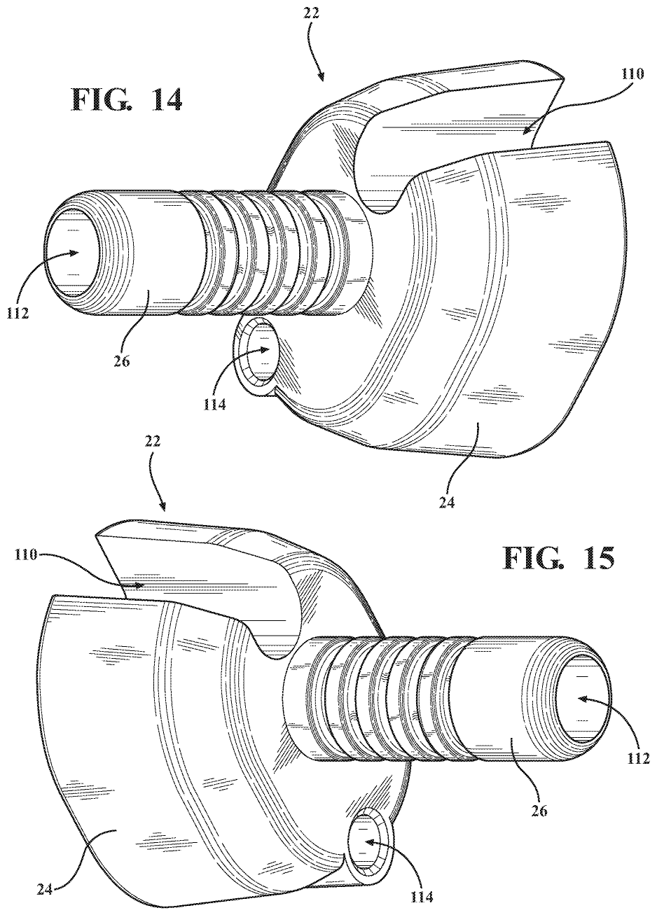

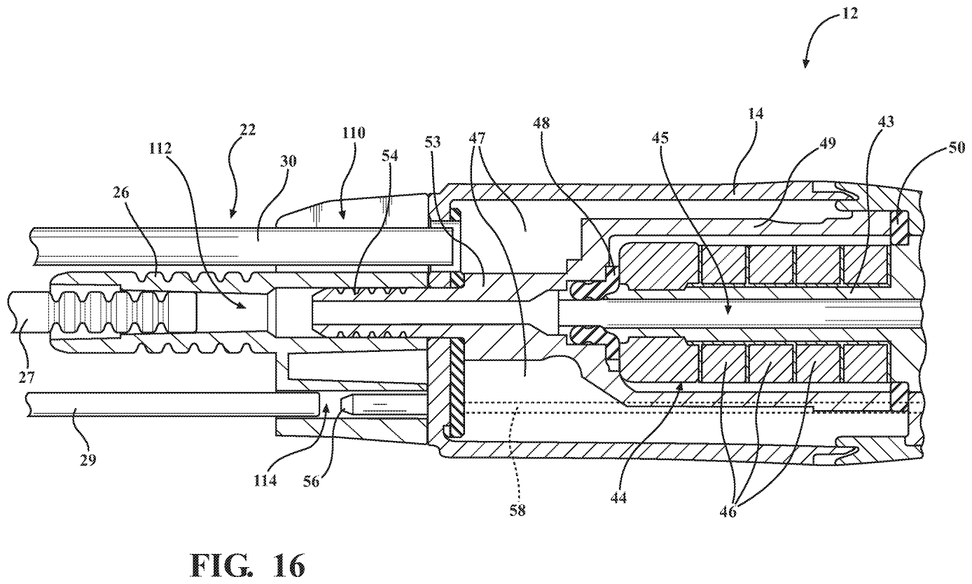

Referring to FIGS. 12-16, an example configuration of the tubing connector 22 for use with the ultrasonic handpiece 12 as part of the ultrasonic surgical handpiece assembly 10 is illustrated. The tubing connector 22 may comprise a base 24 including a distal end and a proximal end. The base 24 may be constructed of a rigid material such as a plastic. Alternatively, the base 24 may be constructed of a flexible material or resilient material such as an elastomer. The tubing connector may be removable from the ultrasonic surgical handpiece and may be shipped with tubing to connect the tubing connector 22 to the cassette for connection of the ultrasonic handpiece to the irrigation source and aspiration source. The tubing connector may be disposed after usage with a particular patient.

The base 24 may be configured to define one or two lumens 112, 114 that extend through the base 24. The lumens 112, 114 may define a fluid passageway through the base 24 of the tubing connector 22 and be configured to be in fluid communication with one or more conduits or lumens through the ultrasonic handpiece 12. For example, a first lumen 112 may be configured to be in fluid communication with the passageway that is formed by the conduit 42 of the horn 40 and the lumen 45 of the transducer 44 when the tubing connector 22 is coupled to the ultrasonic handpiece 12. Additionally, a second lumen 114 may be configured to be in fluid communication with the lumen 59 that is disposed within the ultrasonic handpiece 12 when the tubing connector 22 is coupled to the ultrasonic handpiece 12.

The base 24 may also define a groove 110 in a perimeter of the base 24 that extends between the distal end and the proximal end of the base 24. The groove 110 may be configured to receive the cable 30, wiring harness, or similar conductor that extends proximally from the proximal end of the ultrasonic handpiece 12. For example, the groove 110 of the tubing connector may be configured to receive and partially surround the cable 30, as illustrated in FIG. 1. The groove 110 may be shaped and/or configured to removably couple the base 24 of the tubing connector 22 to the cable 30 via a friction fit or interference fit. For example, the groove 110 may comprise a U-like or crescent shape, wherein the width of the opening of the groove 110 at the surface of the base 24 is less than the maximum width of the cable 30.

The tubing connector 22 or line connector may further comprise a resilient member 26 that extends proximally from the base 24 and is configured to receive the aspiration line 27. The resilient member 26 and base 24 may be formed as a unitary component, wherein the resilient member 26 defines a portion of the lumen 112 through the base 24. The resilient member 26 may be configured such that the aspiration line 27 may be inserted within the lumen 112 at the proximal end of the resilient member 26. The aspiration line 27 may for a friction fit within the lumen of the resilient member. Alternatively, the aspiration line 27 may be coupled to the resilient member 26 using a glue, epoxy, or similar adhesive configured to create a chemical bond.

While not illustrated, it is also contemplated that the resilient member 26 may be removably coupled to the base 24. For example, a distal end of the resilient member 26 may be configured to create a friction fit with the base 24 when inserted in the lumen 112.

While the figures illustrate the aspiration line 27 and/or the irrigation line 29 as being inserted directly within the lumens 112, 114 defined in the base 24 of the tubing connector 22, if is further contemplated that the aspiration line 27 and/or the irrigation line 29 may be coupled to the base 24 by a hose barb or similar fitting. For example, proximal end of a hose barb may be inserted in the opening of the aspiration line 27 and/or the irrigation line 29. The proximal end of the hose barb may then be inserted within the respective lumens 112, 114 that are defined in the base 24. The hose barb(s) may be coupled to the base 24 via friction fit within the lumens 112, 114, or the hose barb(s) may be coupled to the base 24 using an epoxy, glue, sealant, or similar adhesive.

The base 24 may be configured to be removably coupled the proximal end of the ultrasonic handpiece 12 via and interference fit. The interference fit may be created between the coupling feature 56 of the irrigation tube 58 and the coupling portion 54 of the channel 53 and the respective lumens 112, 114. For example, the coupling feature 56 and coupling portion 54 may each comprise a hose barb or similar fitting configured to be inserted in the corresponding lumens 112, 114 to create a friction fit that removably secures the tubing connector 22 to the proximal end of the ultrasonic handpiece 12.

Alternatively, while not illustrated in the figures, it is contemplated that the distal end of the base 24 may comprise a retention feature such as a tab or finger configured to create a snap-fit with the proximal end of the ultrasonic handpiece 12. For example, the retention feature may comprise a tab or protrusion that encircles a perimeter of the distal end of the base 24 and is configured to engage the proximal end of the ultrasonic handpiece 12 to removably couple the tubing connector 22 to the ultrasonic handpiece 12 via a snap fit.

The tubing connector 22 may serve as a means of quickly attaching and detaching both the irrigation line 29 and the aspiration line 27 to the proximal end of the ultrasonic handpiece 12. Furthermore, the tubing connector 22 may removably secure the irrigation line and/or aspiration lines 29, 27 to the ultrasonic handpiece 12 in a manner that reduces the stress on the lines proximate the ultrasonic handpiece 12. The tubing connector 22 may reduce bending and/or kinking of the lines proximate the ultrasonic handpiece 12. For example, the resilient member may comprise a resilient material configured to support the aspiration line 17 and/or resist bending or kinking the aspiration line 17. Furthermore, the proximal offset created by the resilient member extending proximally from the base 24 offsets the location where the aspiration line 27 and the irrigation line 29 are coupled to the base 24. This may prevent tangling of the lines as they drape off the proximal end of the tubing connector 22. This serves to provide support to the lines 27, 29 to resists bending of the line 27, 29 proximate the ultrasonic handpiece 12 to reduce the occurrence of sharp bends in the lines 27, 29 that may restrict flow through the lines 27, 29.

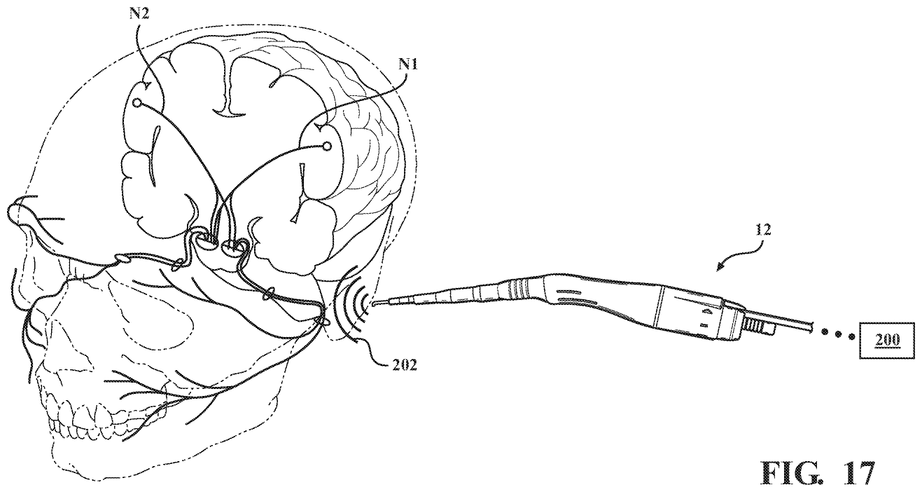

It is contemplated that the ultrasonic handpiece 12 may be used in conjunction with a nerve monitor to provide for detection of nerves in proximity to the ultrasonic handpiece 12. For example, in FIG. 17, a nerve monitor 200 detects cranial nerves N1, N2 in proximity to the ultrasonic handpiece 12. This provides for nerve monitoring functionality while aspirating to reduce the likelihood of inadvertently contacting a nerve with a cutting instrument. This also provides for a single instrument, such that less time is spent by the surgeon switching between a nerve monitor and the ultrasonic handpiece 12. The additional requirements of a separate stimulation probe means an additional instrument in the sterile field, and having to insert and reinsert the stimulation probe and the ultrasonic handpiece 12 are both undesirable. As the surgeon is using the ultrasonic handpiece 12, a nerve monitor stimulation signal 202 is being emitted from the ultrasonic tip 20, even optionally while the ultrasonic tip 20 is active. This allows the surgeon to detect nerves, such as cranial nerves N1, N2, without the use of a separate stimulation probe. This integration can save a great deal of time for a surgeon.

A variety of different nerve monitors 200 can likely be used with the proposed systems. One exemplary nerve monitor device is described in U.S. Pat. No. 9,084,551, which is hereby incorporated by reference in its entirety. Another example is described in U.S. Pat. No. 5,775,331, which is also incorporated by reference in its entirety. The nerve monitor 200 typically includes a stimulator circuit for generating a stimulus, such as the nerve monitor stimulation signal 202 shown in FIG. 17, and a response detection means for detecting and measuring a response and indicating the response of the tissue to the user. The nerve monitor 200 includes an indicator that is controlled based on the response to the tissue. The indicator may be audible via a speaker, tactile, through control of a vibration motor associated with one or more described components, or visual, through control of a light or a suitable display device. The light or display device may be on the nerve monitor 200, on the ultrasonic console 13, on the ultrasonic handpiece assembly 10, or on a device at a remote location.

While the below description describes integration of the nerve monitor functionality into an ultrasonic handpiece, similar implementations could be used for other types of surgical handpieces, such as electrosurgical forceps or probes, rotary handpieces, such has high-speed drills, or end effectors used with robotic tools.

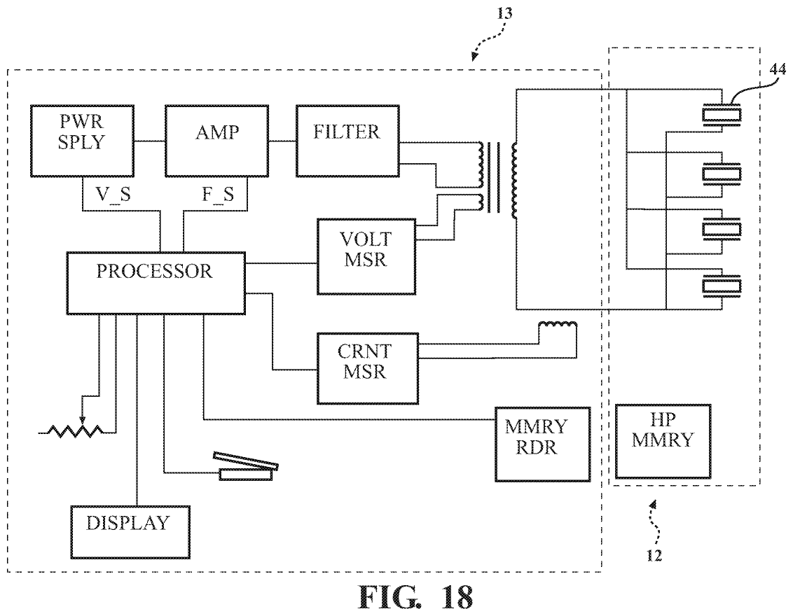

FIG. 18 provides a schematic illustration of the electrical system of the ultrasonic handpiece 12 and related console 13. Additional details can be found in WO2015/021216, which is hereby incorporated by reference in its entirety.

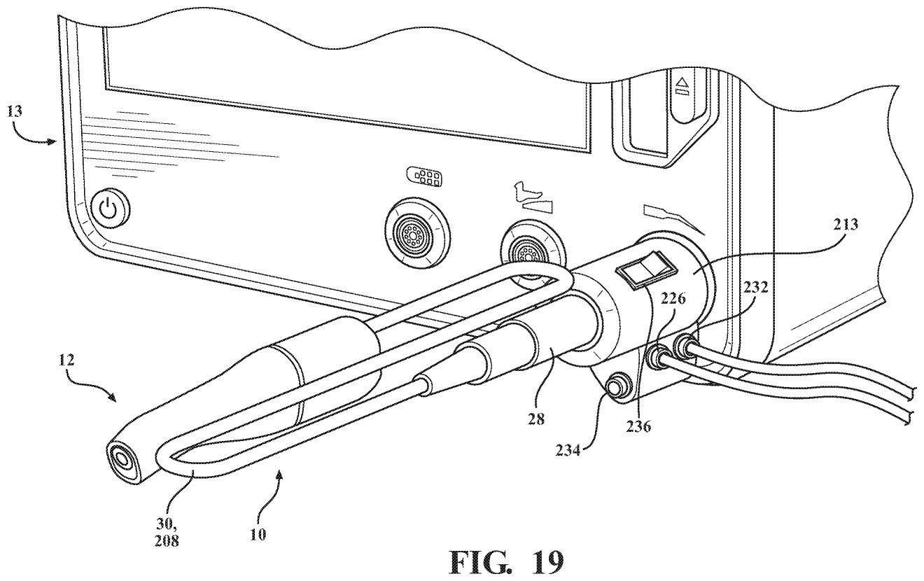

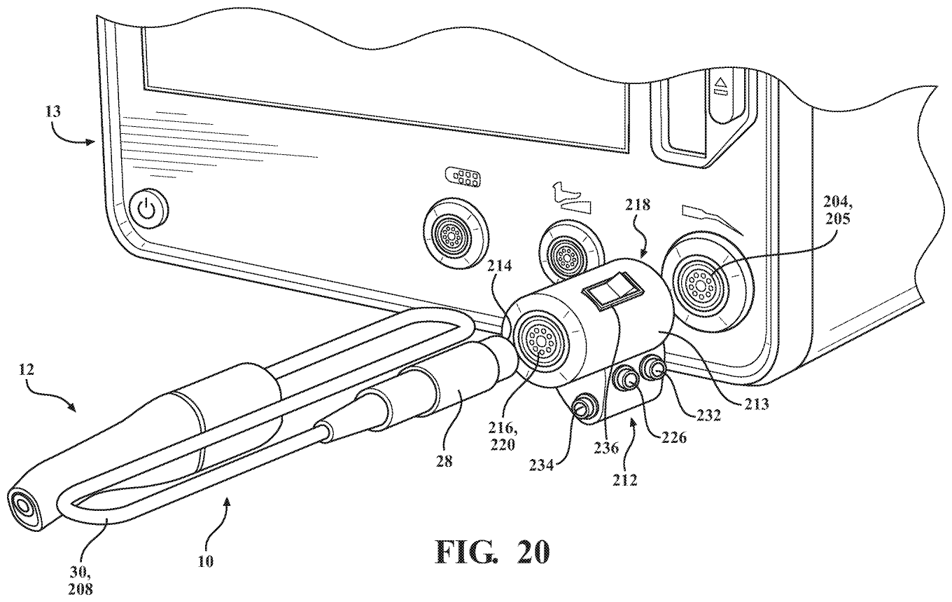

FIGS. 19 and 20 illustrates a first implementation of an integration with a nerve monitor 200, where the nerve monitor 200 is connected to an ultrasonic handpiece assembly 10 via an adapter 212. As shown, the implementation includes an ultrasonic control console 13, an ultrasonic handpiece assembly 10, and an adapter 212. The ultrasonic handpiece assembly 10 includes an ultrasonic handpiece 12, an electrical cord including a conductor 208, and a console connector. The electrical cord may be the previously described cable 30, and the console connector may be the previously described power connector 28. Referring to FIG. 20, the ultrasonic control console 13 includes a handpiece port 204. The system will allow for connection of the ultrasonic handpiece 12 to the nerve monitor 200 or other physiological sensing device. The nerve monitor 200 may be a source of electrical potential.

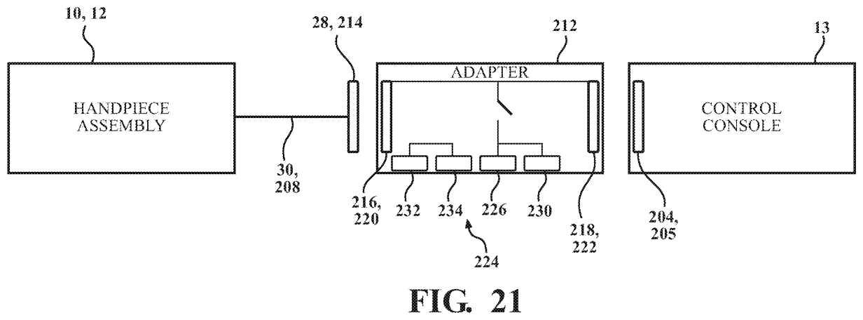

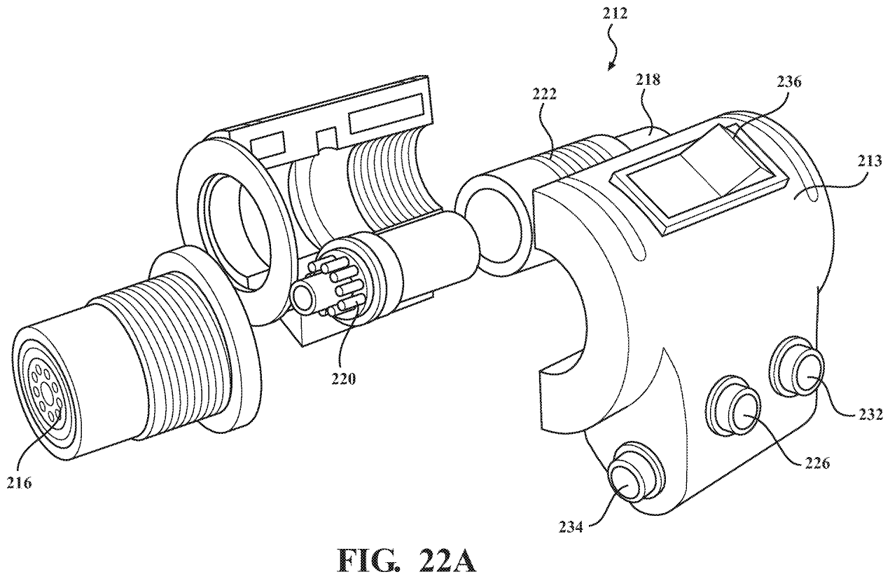

FIG. 21 illustrates terminals of the ultrasonic handpiece assembly 10 and the control console 13. As shown, the console connector 28 of the ultrasonic handpiece assembly 10 includes at least one terminal 214. The console connector 28 is shaped to engage the handpiece port 204 of the control console 13, which includes a terminal 205, during normal usage. The terminals 214, 205 are also shown in FIG. 20.

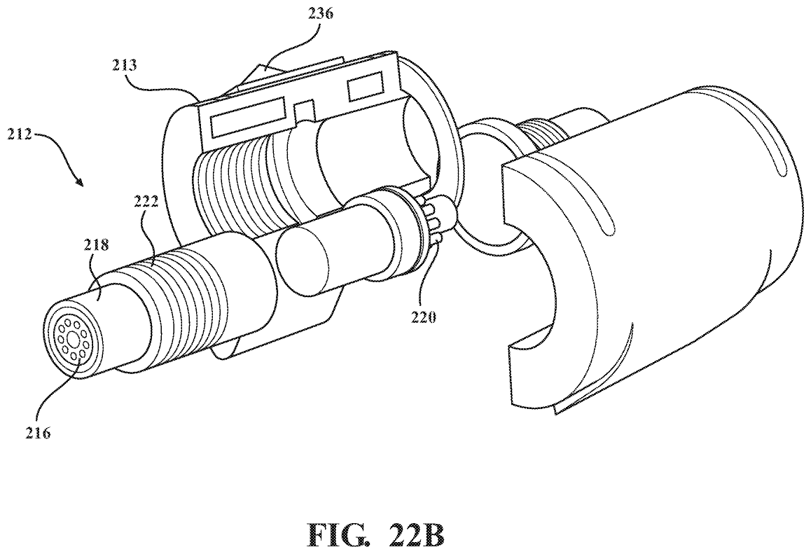

FIG. 21 also illustrates terminals of the adapter 212. As shown, the adapter 212 includes a first connector 216 and a second connector 218, the first connector 216 including at least one terminal 220 and the second connector 218 including at least one terminal 222. The first connector 216 is configured to engage the console connector 28 of the ultrasonic handpiece assembly 10. The first connector 216 of the adapter 212 is configured similar to handpiece port 204 of the control console 13 such that an electrical communication between the adapter 212 and the ultrasonic handpiece assembly 10 can be established. The adapter 212 further includes a second connector 218 for engaging the handpiece port 204 of the control console 13. The terminal 214 of the console connector 28 is configured to engage the terminal 220 of the first connector 216, allowing for electrical communication between the ultrasonic handpiece assembly 10 and the adapter 212. The terminal 222 of the second connector 218 is configured to engage the terminal 205 of the control console 13, allowing for electrical communication between the adapter 212 and the control console 13. The first connector 216 and the terminal 220 are further shown in FIG. 22A and the second connector 218 and the terminal 222 are further shown in FIG. 22B.

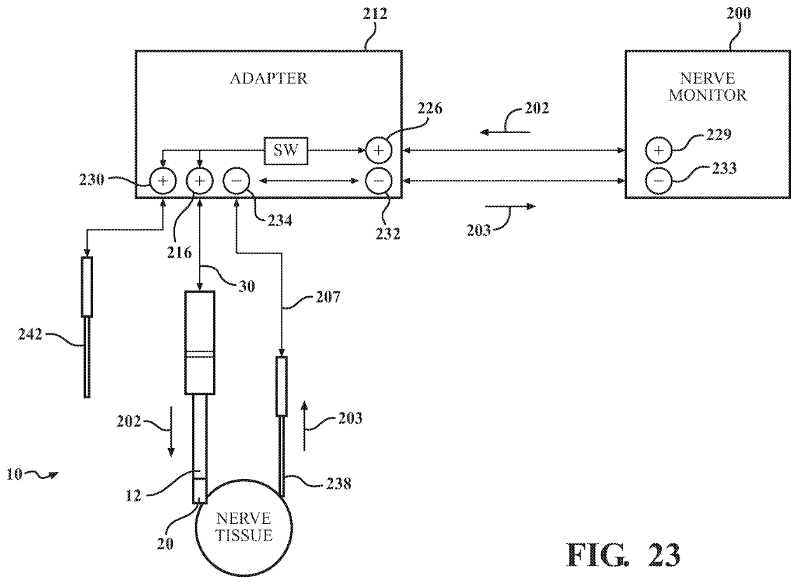

Also shown in FIG. 21, adapter 212 also includes at least one terminal 224 configured to engage various connectors from the nerve monitor 200. For example, the at least one terminal 224 may include an input terminal 226. The input terminal 226 is further shown in FIG. 22A. As shown in FIG. 23, the input terminal 226 may be configured to receive a terminal 229 from the nerve monitor 200. The adapter 212 may utilize a jump conductor 228 (shown in FIG. 21) to establish an electrical communication between the input terminal 226 and the adapter first connector 216. In this way, electrical communication can be established between the nerve monitor 200 and the ultrasonic handpiece assembly 10.

In certain configurations, the at least one terminal 224 of the adapter 212 may include additional terminals to enhance functionality. For example, as shown in FIG. 21, the adapter 212 may additionally include a reference input terminal 232 and a reference output terminal 234. The reference input terminal 232 and the reference output terminal 234 are further shown in FIG. 22A. Referring to FIG. 23, reference input terminal 232 may receive a terminal 233 of the nerve monitor 200 to receive a reference signal. Also shown, the reference output terminal 234 may be connected to an auxiliary stimulation probe 238. The reference input terminal 232 and the reference output terminal 234 allow the adapter 212 to serve as a pass through for the reference signal to be channeled from the auxiliary stimulation probe 238, through the adapter 212, and back to the nerve monitor 200. This functionality allows for several cords associated with the procedure, such as the cable 30 from the ultrasonic handpiece 12 and a cord 207 extending from an auxiliary stimulator probe, to extend from a single location to the surgical site, i.e., the adapter 212, as opposed to a first cord extending from the nerve monitor 200 to an auxiliary stimulation probe and a second cord extending from the control console 13 to the ultrasonic handpiece 12.

FIG. 23 illustrates an operation of the adapter 212 and the nerve monitor 200. As shown, a circuit is completed between the ultrasonic handpiece 12 and the auxiliary stimulation probe 238 when the ultrasonic tip 20 and a tip 240 of the auxiliary stimulation probe 238 are in electrical contact with nerve tissue. In this way, the nerve monitor stimulation signal 202 transmitted from the nerve monitor 200 may be emitted from the ultrasonic tip 20 to the nerve tissue. The nerve monitor 200 may then detect and measure a response of the nerve tissue by measuring a response signal 203 to detect a presence of a nerve in proximity to the ultrasonic handpiece 12.

In some instances, such as the instances of FIGS. 21 and 23, the adapter 212 may optionally include a switch 236. The switch 236 may be placed in electrical communication between the input terminal 226 and the first connector 216 to control transmission of the nerve stimulation signal 202 from the nerve monitor 200 through the ultrasonic handpiece 12. In instances where the switch 236 is open, the nerve stimulation signal 202 would not be transmitted from the nerve monitor 200 and through the ultrasonic handpiece 12. In instances where the switch 236 is closed, the nerve stimulation signal 202 may be transmitted from the nerve monitor 200 through the ultrasonic handpiece 12. It may be advantageous to stop the nerve stimulation signal 202 from being transmitted through the ultrasonic handpiece 12 during certain aspects of a procedure, such as during certain times during an open craniotomy, where the nerve stimulation signal 202 can be bothersome to a patient. The switch 236 may be digital or electrical. The switch 236 may be accessible on an external surface of the adapter 212, such as on a housing 213 of the adapter 212, as shown in FIGS. 19, 20, and 22A.

In some instances, such as the instance of FIGS. 21 and 23, the terminals 224 of the adapter 212 may optionally include an output terminal 230. As shown in FIG. 23, the output terminal 230 may be connected to a secondary probe 242. Also shown, the output terminal 230 may be in electrical communication with the input terminal 226 from the nerve monitor 200. The secondary probe 242 may be used in addition to or as an alternative to the ultrasonic handpiece 12 when detecting a presence of a nerve. The output terminal 230 serves as another way for surgeons to deliver stimulating energy to surgical locations.

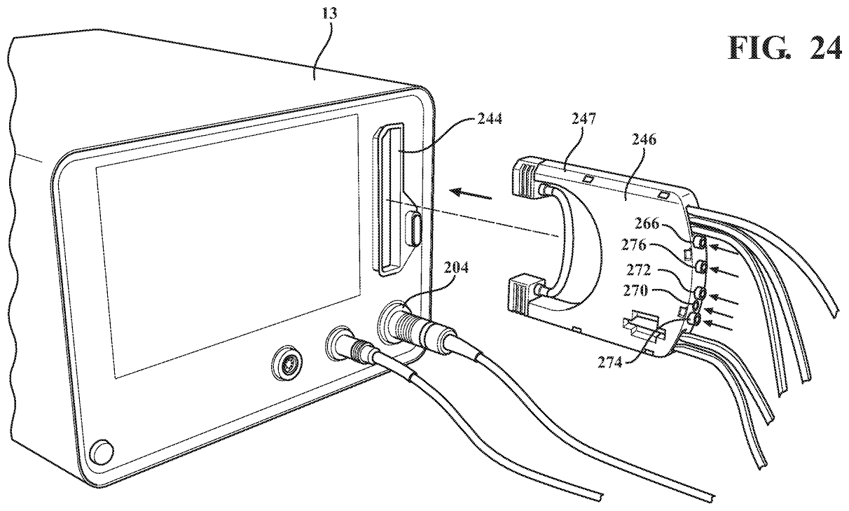

FIGS. 24-27 provide for another potential implementation of the integration of nerve monitoring functionality with the ultrasonic handpiece assembly 10. In such an implementation, a cassette/cassette receptacle interface may provide for a connection between the nerve monitor 200 and the ultrasonic handpiece 12 or other type of handpiece. As shown, the previously described control console 13 may define a cassette receptacle 244, which may accept a cassette 246. The cassette 246 may be any suitable cassette, such as an irrigation cassette, an aspiration cassette, or a dual-purpose irrigation/aspiration cassette. While not to be limited to the cassette construction described therein, an exemplary cassette/cassette receptacle construction is described in WO2020/068509, which is hereby incorporated by reference in its entirety.

As described above with respect to the adapter, the cassette-style implementation may be utilized with other surgical consoles and other surgical handpieces other than those for ultrasonic surgical devices, such as for RF surgical devices, high-speed drills, and other cutting or resection devices.

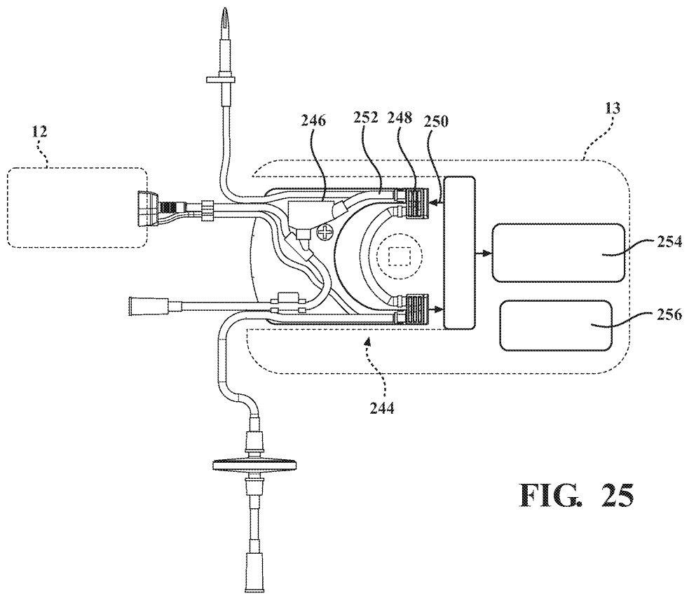

With respect to FIG. 25, the cassette 246 may include a cassette fluid port 248 that is shaped to engage a console fluid port 250 of the control console 13. The cassette 246 may also include a surgical tube 252 in communication with the cassette fluid port 250. The other end of the surgical tube 252 may extend to the ultrasonic surgical handpiece 12. The surgical tube 252 may also be referred to as an irrigation line or an aspiration line. The console fluid port 250 may be in fluid communication with an aspiration pump 254 or an irrigation pump 256 of the control console 13, depending on the type of cassette 246. In this way, the two fluid ports 248, 250 are configured to establish a fluid connection between one of the pumps 254, 256 and the surgical tube 252 when the cassette 246 is inserted into the cassette receptacle 244.

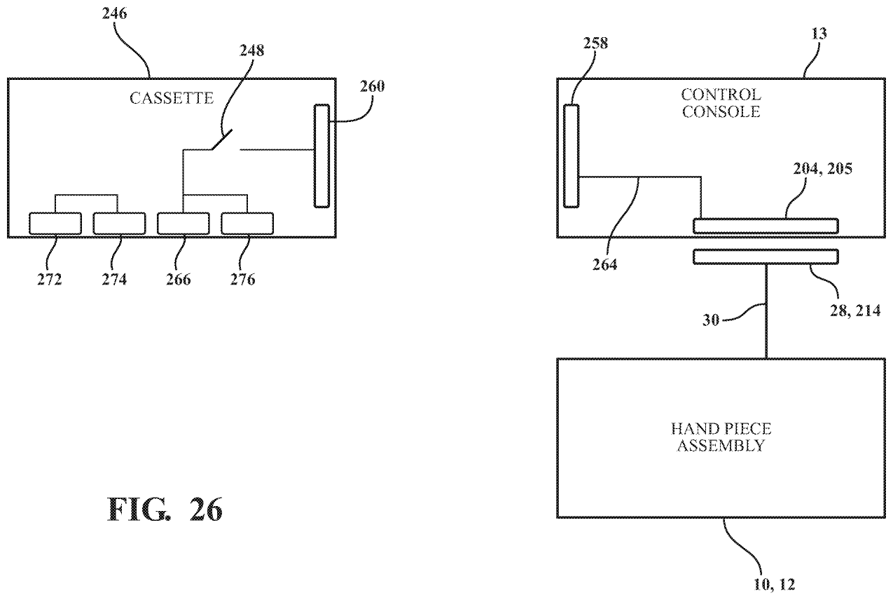

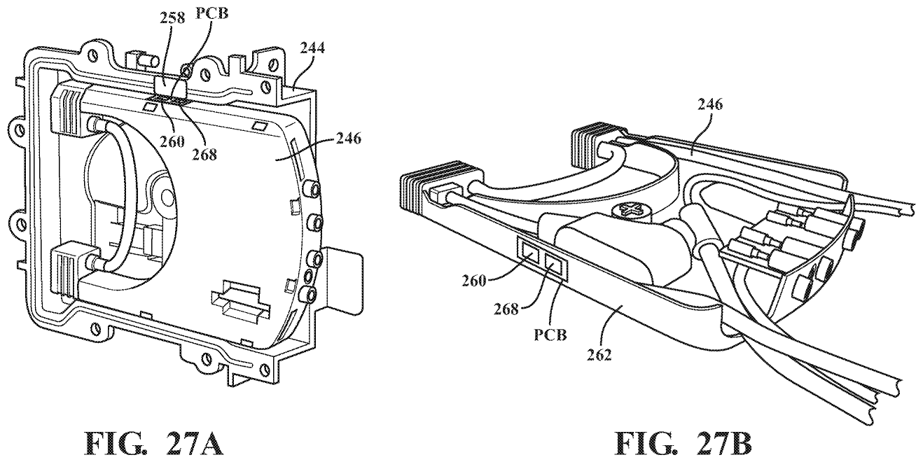

Referring now to FIG. 26, the control console 13 may include a cassette receiver terminal 258 and the cassette 246 may include a cassette terminal 260. As shown in FIG. 27A, the control console 13 and the cassette 246 may be configured such that an electrical connection is established between the cassette receiver terminal 258 and the cassette terminal 260 when the cassette 246 is fully inserted within the cassette receiver 244 of the control console 13. The cassette receiver terminal 258 may be implemented as a roller pin. As shown in FIG. 27, the cassette terminal 260 may be defined on a printed circuit board PCB. This printed circuit board PCB may define a portion of an external surface of the cassette 246. For example, the printed circuit board PCB may define a portion of a housing 262 of the cassette 246.

As shown in FIG. 24, the control console 13 may further include the previously described handpiece port 204. The handpiece port 204 may be configured to be engage the previously described console connector 28 of the ultrasonic handpiece assembly 10 such than when the console connector 28 engages the handpiece port 204, an electrical connection is established between the console terminal 205 and the terminal 214 of the console connector 28, establishing an electrical connection between the control console 13 and the handpiece assembly 10. The control console 13 may also include a jump conductor 264 or other conductive pathway between the cassette receiver 244 and the console terminal 205 such that electrical communication is established between the ultrasonic handpiece assembly 10 and the cassette receiver terminal 244. The jump conductor 264 may be implemented using an SMA connector.

The cassette 246 may include several other components. The cassette 246 may include an input terminal 266 for receiving a connector from the nerve monitor 200. A conductive path extends from this input terminal 266 to the cassette terminal 260 such that the nerve stimulation signal 202 from the nerve monitor 200 can extend from the nerve monitor 200 the handpiece assembly 10 through the cassette 246 and control console 13.

The cassette 246 may also include a switch 268 in electrical communication between the cassette terminal 260 and the input terminal 266. The switch 268 may selectively control whether the nerve stimulation signal 202 is transmitted from the nerve monitor 200 to the ultrasonic handpiece assembly 10. As shown in FIG. 27B, the switch 268 may be defined on the printed circuit board PCB of the cassette 246.

In some configurations, such as the configuration shown in FIG. 24, the cassette may include a separate connector 270 for engaging an external switch, such as a footswitch. The footswitch may be used in such a configuration to control the switch 268 of the cassette. Alternatively, the switch 268 may be engageable by a user on the surface of the cassette or at some other location.

Referring to FIG. 26, the cassette may further include a reference input terminal 272 and a reference output terminal 274. The reference input terminal 272 is configured to receive a connector from the nerve monitor 200 for providing a reference signal. The reference output terminal 274 is for receiving a connector of a reference probe or other reference device. Similar to the advantage described in the configuration including the adapter 212, the reference input terminal 272 and the reference output terminal 274 provide fewer origin locations for medical lines extending to the surgical site. This is because a line associated with a reference probe can be routed from the cassette 246 to the surgical site, and a line associated with the ultrasonic handpiece assembly 10 can be routed from the cassette 246 to the ultrasonic handpiece assembly 10 located at the surgical site.

With further reference to FIG. 26, the cassette may further include an output terminal 276 for a secondary or auxiliary stimulation probe. The output terminal 276 may be in electrical communication with the input terminal 266 from the nerve monitor 200. The output terminal 276 serves as another way for surgeons to deliver stimulating energy to surgical locations.

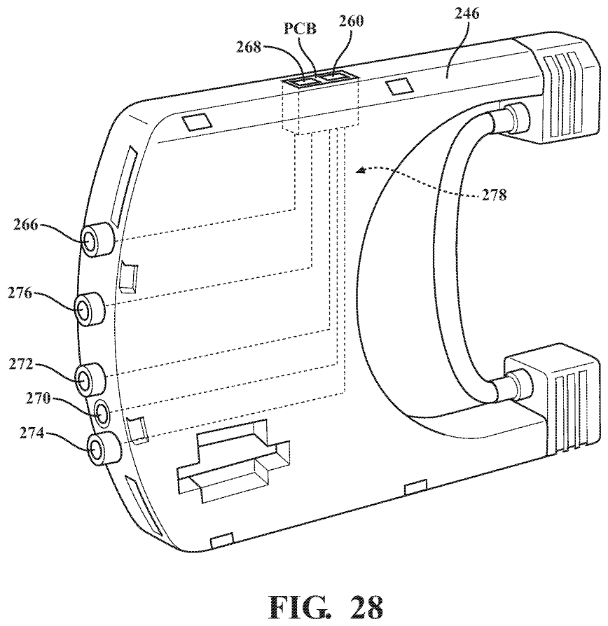

As indicated by FIG. 28, the printed circuit board PCB may serve as a hub for the various terminals 266, 270, 274, 272 and for the connector 270 to route signals as necessary. The conductors 278 extending from the various terminals 266, 270, 274, 272 and from the connector 270 may be routed within the cassette 244.

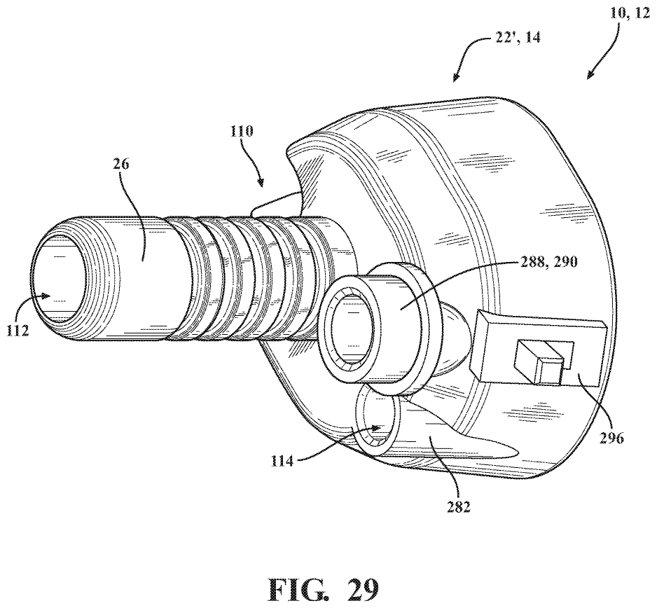

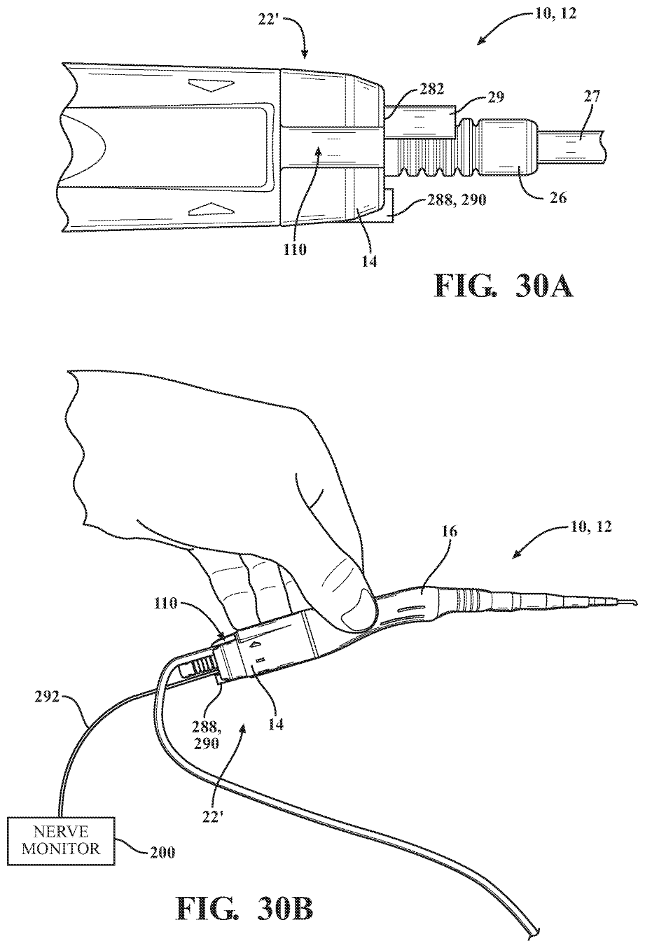

Referring to FIG. 29, in another potential implementation, the ultrasonic handpiece assembly 10 may feature a line connector in its proximal end 14. The line connector may be an instance of the previously described tubing connector 22 and is referred to herein as “line connector 22′”. The line connector 22′ may include an irrigation tubing coupler 282, an aspiration tubing coupler, which may be the previously described resilient member 26, and a void for receiving a power cord, where the void may be the previously described groove 110 for receiving the cord 30. Additionally, the line connector 22′ may include a receptacle 288 including a terminal 290 for receiving an input signal, such as the nerve stimulation signal 202, from the nerve monitor 200. As shown in FIG. 30A, the irrigation line coupler 282 defines the irrigation lumen 114 and may be configured to releasably engage an irrigation line 29. Also shown, the aspiration line coupler 26 defines the aspiration lumen 112 and may be configured to releasably engage an aspiration line 27. Referring to FIG. 30B, the receptacle 288 is shaped to receive a connector 292 extending from the nerve monitor 200, such that the terminal 290 of the receptacle 288 can receive an input signal from the nerve monitor 200. Additionally, the void 110 may receive the cord 30. As shown in FIG. 30B, this implementation provides a convenient location for connecting the ultrasonic handpiece 12 to the nerve monitor 200, which does not interfere with handling of the ultrasonic handpiece 12.

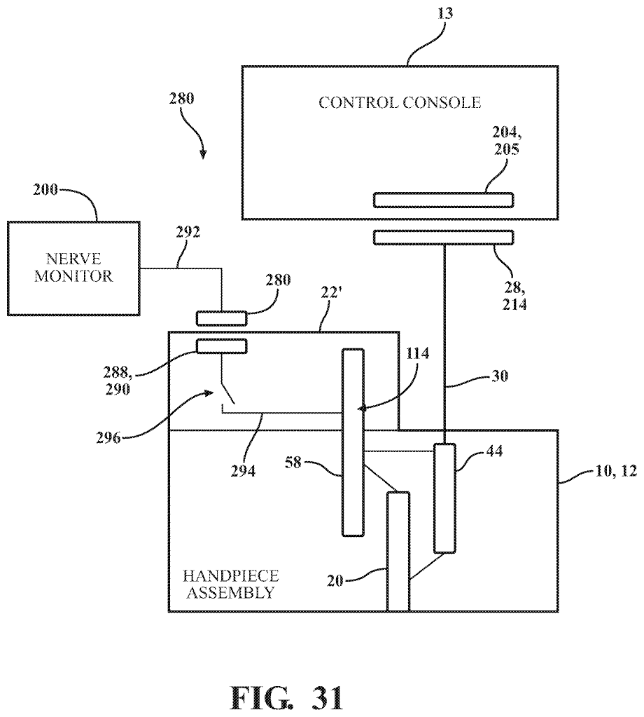

Referring to FIG. 31, an electrical schematic of the implementation of FIG. 29-31B is shown. In this implementation, the receptacle 288 includes the input terminal 290, which receives an input signal from the nerve monitor 200. The ultrasonic handpiece 12 includes an irrigation conduit 58 and an electromechanical transducer 44. The terminal 290 within the line connector 22′ is placed in electrical communication with the irrigation conduit 58 and/or the electromechanical transducer 44 when the line connector is coupled to the proximal end of the handpiece and optionally, when saline or other conductive fluid flows through the irrigation conduit. More particularly, the ultrasonic tip 20 is in electrical communication with the irrigation conduit 58 and/or the electrical mechanical transducer 44 via a jump conductor 294. This is because the irrigation conduit 58 within the handpiece 12 may be formed from a metallic component, such as stainless steel, or because the irrigation conduit may contain a conductive fluid. Based on this configuration, an input signal transmitted from the nerve monitor 200, such as the nerve stimulation signal 202, is provided to the tip 20 of the ultrasonic handpiece 12 via the terminal 290 within the receptacle 288.

In some instances, the line connector 22′ may include a switch 296. The switch 296 may be placed in electrical communication between the terminal 290 and the irrigation conduit 58 to control transmission of the nerve stimulation signal 202 from the nerve monitor 200 through the ultrasonic handpiece 12. The switch 296 may be accessible on an external surface of the line connector 22′, as shown in FIG. 29.

In some instances, the line connector 22′ may be detachable from the ultrasonic handpiece 12. For example, the line connector 22′ may be a disposable component that may be coupled to the ultrasonic handpiece 12 in implementations where the nerve monitor 200 is integrated. In the instance of FIG. 31, the line connector 22′ may be detachable from the ultrasonic handpiece 12. In such instances, the line connector 22′ may include the jump conductor 294, and the jump conductor 294 may be disposed in electrical communication between the terminal 290 and the irrigation conduit 58 via the lumen 114. In this way, the nerve stimulation signal 202 may be conducted to the irrigation conduit 58 via irrigation fluid flowing through the lumen 114. In alternative implementations, the jump conductor may be used to place the nerve monitor in communication with the aspiration line or conduit with the handpiece assembly.

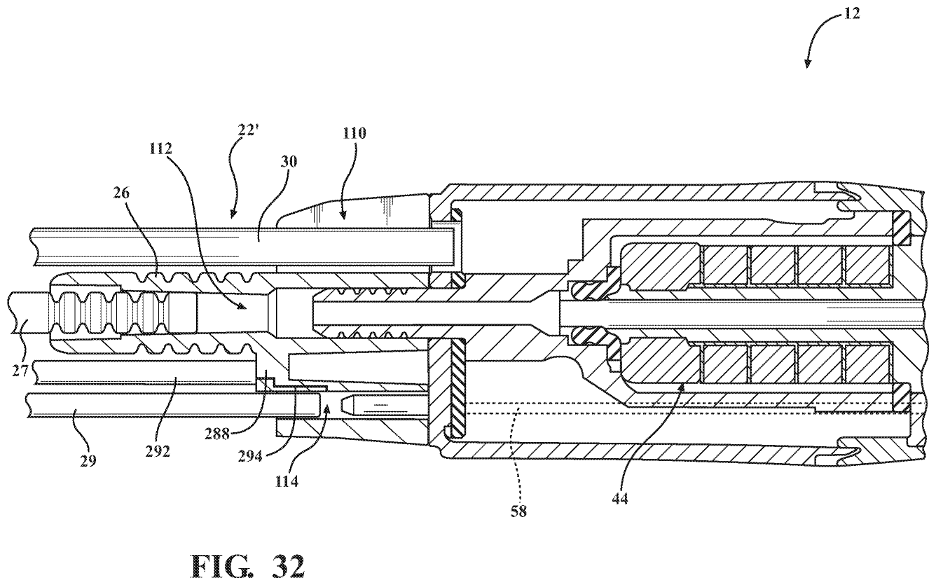

A cut-away sectional view of the ultrasonic handpiece 12 and the line connector 22′ are shown in FIG. 32 to further illustrate that the jump conductor 294 may be in electrical communication with the lumen 114. Because the lumen 114 is in fluid communication with the ultrasonic tip 20 of the ultrasonic assembly 10, an input signal from the nerve monitor 200 can travel to the ultrasonic tip 20 via the fluid within the lumen 114 or via the tube that defines the lumen 114. It should be appreciated a terminal could be used in combination with the jump conductor 294 to facilitate transmission of the input signal to the tip 20.

It should be noted that FIG. 31 optionally omits components of the control console 13, the line connector 22′, and the ultrasonic handpiece assembly 10 for the purposes of illustration. For example, the aspiration lumen 112 of the line connector 22′ is optionally omitted for purposes of illustration.

Several embodiments have been discussed in the foregoing description. However, the embodiments discussed herein are not intended to be exhaustive or limit the invention to any particular form. For example, while the example configurations describe the surgical instrument as an ultrasonic handpiece, it is further contemplated that the features and concepts described with regard to the ultrasonic handpiece may be applied to other medical or surgical instruments. This similarly applies to the ultrasonic tip 20, which may further include blades, drill bits, rotating burs, open-window shavers, and the like. The terminology which has been used is intended to be in the nature of words of description rather than of limitation. Many modifications and variations are possible in light of the above teachings and the invention may be practiced otherwise than as specifically described.

1. An ultrasonic handpiece assembly comprising:
a housing;
an electromechanical transducer at least partially disposed within the housing;
a tip operatively coupled to the electromechanical transducer;
an electrical cord having a console connector, the electrical cord including a first conductor, the first conductor being in electrical communication with the electromechanical transducer;
a line connector comprising:
a first tubing coupler configured to receive an irrigation line;
a second tubing coupler configured to receive an aspiration line; and
a receptacle including a terminal, the receptacle being configured to receive a line extending from a nerve monitor,
wherein the terminal is in electrical communication with the tip.

2. The ultrasonic handpiece assembly of claim 1, wherein the line connector is formed from a resilient material.

3. The ultrasonic handpiece assembly of claim 1, wherein the housing includes an irrigation conduit, the terminal being in electrical communication with the irrigation conduit.

4. The ultrasonic handpiece assembly of claim 3, wherein the line connector comprises a jump conductor arranged to place the terminal in electrical communication with the irrigation conduit.

5. The ultrasonic handpiece assembly of claim 3, wherein the line connector comprises a switch, the switch being in electrical communication with the terminal, and operable to place the terminal out of electrical communication with the irrigation conduit.

6. The ultrasonic handpiece assembly of claim 1, wherein the line connector defines a void for receiving a portion of the electrical cord.

7.-12. (canceled)

13. An ultrasonic handpiece assembly comprising:
a housing;
an electromechanical transducer at least partially disposed within the housing;
a tip operatively coupled to the electromechanical transducer; and
an electrical cord having a console connector, the electrical cord including a first conductor, the first conductor being in electrical communication with the electromechanical transducer and the tip,
wherein the console connector includes a first terminal in electrical communication with the first conductor, the first terminal for engaging an ultrasonic control console, a second terminal for engaging a source of electrical potential of a nerve monitor, and a jump conductor arranged to place the second terminal in electrical communication with the first conductor.

14. The ultrasonic handpiece assembly of claim 13, wherein the console connector includes a third terminal, the third terminal for establishing a connection with a reference probe.

15. The ultrasonic handpiece assembly of claim 13, wherein the console connector includes a fourth terminal, the fourth terminal for establishing a connection with a stimulator probe, the stimulator probe being separate from the ultrasonic handpiece assembly.

16. The ultrasonic handpiece assembly of claim 13, further comprising a switch, the switch being in electrical communication with the second terminal, and operable to place the second terminal out of communication with the first conductor.

17. The ultrasonic handpiece assembly of claim 16, wherein the switch is a footswitch.

18. The ultrasonic handpiece assembly of claim 16, wherein the console connector includes the switch.

19. An ultrasonic surgical system comprising:
an ultrasonic handpiece assembly including:
a housing;
an electromechanical transducer at least partially disposed within the housing;
a tip operatively coupled to the electromechanical transducer; and
an electrical cord having a console connector, the electrical cord including a first conductor, the first conductor being in electrical communication with the electromechanical transducer and the tip;
wherein the console connector includes a first terminal in electrical communication with the first conductor; and
an adapter for connecting the ultrasonic handpiece assembly to a nerve monitor, the adapter comprising a second terminal for engaging an ultrasonic control console, a third terminal for engaging a source of electrical potential of a nerve monitor, and a jump conductor arranged to place the third terminal in electrical communication with the second terminal;
wherein, upon connection of the console connector to the adapter, electrical communication is established from the third terminal to the first terminal.

20. The system of claim 19, wherein the adapter includes a fourth terminal, the fourth terminal for establishing a connection with a reference input from the nerve monitor and a sixth terminal for establishing a connection with a reference probe.

21. The system of claim 19, wherein the adapter includes a fourth terminal, the fourth terminal for establishing a connection with a stimulator probe, the stimulator probe being separate from the ultrasonic handpiece assembly.

22. The system of claim 19, further comprising a switch, the switch being in electrical communication with the third terminal, and operable to place the second terminal out of communication with the first conductor.

23. The system of claim 22, wherein the switch is a footswitch.

24. The system of claim 22, wherein the adapter includes the switch.

25.-40. (canceled)
